(19) United States
(12) Patent Application Publication
(10) Pub. No.: US 2023/0335240 A1
(43) Pub. Date: Oct. 19, 2023

(54) PRESYMPTOMATIC DISEASE DIAGNOSIS DEVICE, PRESYMPTOMATIC DISEASE DIAGNOSIS METHOD, AND TRAINED MODEL GENERATION DEVICE

(71) Applicant: Mitsubishi Electric Corporation, Tokyo (JP)

(72) Inventors: Tsutomu MATSUBARA, Tokyo (JP); Masato HIRAI, Tokyo (JP); Hitoshi YOSHIZAWA, Tokyo (JP)

(73) Assignee: Mitsubishi Electric Corporation, Tokyo (JP)

(21) Appl. No.: 18/213,291

(22) Filed: Jun. 23, 2023

Related U.S. Application Data
(63) Continuation of application No. PCT/JP2021/001142, filed on Jan. 15, 2021.

(51) Int. Cl. G16H 10/60; G16H 50/20

(52) U.S. Cl. CPC G16H 10/60; G16H 50/20

(57) ABSTRACT

A presymptomatic disease diagnosis device is configured to include: a log acquiring unit to acquire a log indicating a change in a body of a person to be diagnosed for a presymptomatic disease; a nursing care data acquiring unit to acquire nursing care data indicating a nursing care content for the person to be diagnosed; and a presymptomatic disease diagnosing unit to give the log acquired by the log acquiring unit and the nursing care data acquired by the nursing care data acquiring unit to a trained model and acquire, from the trained model, diagnostic data indicating a presymptomatic disease possibly occurring in the person to be diagnosed.

### CROSS-REFERENCE TO RELATED APPLICATION

The present application is a bypass-continuation of International Patent Application No. PCT/JP2021/001142, filed Jan. 15, 2021, the entire contents of which are incorporated herein by reference.

### TECHNICAL FIELD

The present disclosure relates to a presymptomatic disease diagnosis device, a presymptomatic disease diagnosis method, and a trained model generation device.

### BACKGROUND ART

In general, a doctor diagnoses that a disease has developed in a person to be diagnosed when finding of abnormality in a blood test result of the person to be diagnosed, an image test result of the person to be diagnosed, or the like. Even if there is no obvious finding of abnormality in the test result such as the blood test result, if there is a sign of abnormality in the test result, there is a possibility that a presymptomatic disease, which is a pre-stage state of the disease, is occurring in the person to be diagnosed. Therefore, a doctor may follow up the change in test results in the person to be diagnosed.

Meanwhile, as a technique for predicting the occurrence of a specific event that can occur in the future in a person to be diagnosed, Patent Literature 1 discloses an event prediction system that observes a change in body motion data indicating acceleration of the body of the person to be diagnosed and predicts the occurrence of the specific event on the basis of the change in the body motion data. The person to be diagnosed is a resident of a nursing home or a patient who is hospitalized in a hospital. The specific event is an event in which the person to be diagnosed falls during walking. The event that the person to be diagnosed falls during walking may occur due to a decrease in the motor function of the person to be diagnosed.

### CITATION LIST

### Patent Literature

Patent Literature 1: JP 2019-155071 A

### SUMMARY OF INVENTION

### Technical Problem

A doctor may be able to discover a presymptomatic disease occurring in a person to be diagnosed by observing a change in test results in the person to be diagnosed. The presymptomatic disease state includes not only a state in which an abnormal finding is observed in the test result (hereinafter referred to as “abnormal finding present state”) even if the person to be diagnosed does not have the subjective symptom but also a state in which the person to be diagnosed has the subjective symptom but no abnormal finding is observed in the test result (hereinafter referred to as “abnormal finding absent state”).

The presymptomatic disease that can be found by the doctor's follow-up of the change in test results is the presymptomatic disease in the abnormal finding present state, and there is a problem that the doctor cannot find the presymptomatic disease in the abnormal finding absent state even if the doctor's follow-up of the change in test results.

Even if the event prediction system disclosed in Patent Literature 1 can notify the doctor of the prediction result of the occurrence of the specific event, the prediction result is a prediction result as to whether or not the specific event occurs, and is not a test result indicating deterioration in motor function. For this reason, the doctor cannot diagnose the presymptomatic disease even with reference to the prediction result.

The present disclosure has been made to solve the problems as described above, and an object thereof is to obtain a presymptomatic disease diagnosis device and a presymptomatic disease diagnosis method capable of diagnosing a presymptomatic disease in the abnormal finding absent state.

### Solution to Problem

A presymptomatic disease diagnosis device according to the present disclosure includes: processing circuitry performing a process to: acquire a log indicating a change in a body of a person to be diagnosed; acquire nursing care data indicating a nursing care content for the person to be diagnosed; and give the log acquired and the nursing care data acquired to a trained model and acquire, from the trained model, diagnostic data indicating presymptomatic diseases including a state of no diagnosis of there being abnormality, possibly occurring in the person to be diagnosed.

### Advantageous Effects of Invention

According to the present disclosure, it is possible to diagnose a presymptomatic disease in the abnormal finding absent state.

### BRIEF DESCRIPTION OF DRAWINGS

FIG. 1 is a configuration diagram illustrating a presymptomatic disease diagnosis device 1 according to a first embodiment.

FIG. 2 is a hardware configuration diagram illustrating hardware of the presymptomatic disease diagnosis device 1 according to the first embodiment.

FIG. 3 is a hardware configuration diagram of a computer in a case where the presymptomatic disease diagnosis device 1 is implemented by software, firmware, or the like.

FIG. 4 is a configuration diagram illustrating a trained model generation device 3 according to the first embodiment.

FIG. 5 is a hardware configuration diagram illustrating hardware of the trained model generation device 3 according to the first embodiment.

FIG. 6 is a hardware configuration diagram of a computer in a case where the trained model generation device 3 is implemented by software, firmware, or the like.

FIG. 7 is a flowchart illustrating a presymptomatic disease diagnosis method which is a processing procedure of the presymptomatic disease diagnosis device 1 illustrated in FIG. 1.

FIG. 8 is a flowchart illustrating a trained model generation method which is a processing procedure of the trained model generation device 3 illustrated in FIG. 4.

FIG. 9 is an explanatory diagram illustrating a diagnostic result of a presymptomatic disease for a person to be diagnosed.

FIG. 10 is an explanatory diagram illustrating information on a presymptomatic disease possibly occurring in the person to be diagnosed.

FIG. 11 is a configuration diagram illustrating a presymptomatic disease diagnosis device 1 according to a second embodiment.

FIG. 12 is a hardware configuration diagram illustrating hardware of the presymptomatic disease diagnosis device 1 according to the second embodiment.

FIG. 13 is a configuration diagram illustrating a trained model generation device 3 according to the second embodiment.

FIG. 14 is a hardware configuration diagram illustrating hardware of the trained model generation device 3 according to the second embodiment.

FIG. 15 is an explanatory diagram illustrating information on presymptomatic disease possibly occurring in a person to be diagnosed.

FIG. 16 is a configuration diagram illustrating a presymptomatic disease diagnosis device 1 according to a third embodiment.

FIG. 17 is a hardware configuration diagram illustrating hardware of the presymptomatic disease diagnosis device 1 according to the third embodiment.

FIG. 18 is an explanatory diagram illustrating an example of a place where an abnormality occurs in a facility.

FIG. 19 is an explanatory diagram illustrating a list of persons to be diagnosed whose vitals are abnormal.

FIG. 20 is a configuration diagram illustrating a presymptomatic disease diagnosis device 1 according to a fourth embodiment.

FIG. 21 is a hardware configuration diagram illustrating hardware of the presymptomatic disease diagnosis device 1 according to the fourth embodiment.

FIG. 22 is an explanatory diagram illustrating a display example of a position where a sensor 15a-n is installed and environment data output from the sensor 15a-n.

FIG. 23 is a configuration diagram illustrating a presymptomatic disease diagnosis device 1 according to a fifth embodiment.

FIG. 24 is a hardware configuration diagram illustrating hardware of the presymptomatic disease diagnosis device 1 according to the fifth embodiment.

FIG. 25 is an explanatory diagram illustrating movement of a skeleton of a person to be diagnosed.

FIG. 26 is a configuration diagram illustrating a presymptomatic disease diagnosis device 1 according to a sixth embodiment.

FIG. 27 is a hardware configuration diagram illustrating hardware of the presymptomatic disease diagnosis device 1 according to the sixth embodiment.

FIG. 28 is an explanatory diagram illustrating a change in a sleeping state and an operation status of an air conditioner.

### DESCRIPTION OF EMBODIMENTS

In order to explain the present disclosure in more detail, embodiments for carrying out the present disclosure will be described below with reference to the accompanying drawings.

### First Embodiment

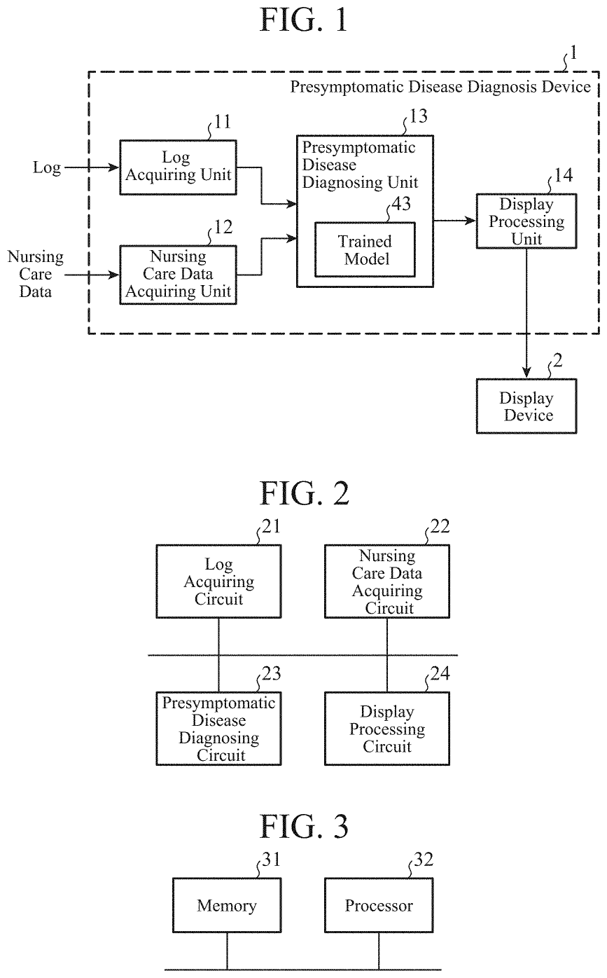

FIG. 1 is a configuration diagram illustrating a presymptomatic disease diagnosis device 1 according to a first embodiment.

FIG. 2 is a hardware configuration diagram illustrating hardware of the presymptomatic disease diagnosis device 1 according to the first embodiment.

The presymptomatic disease diagnosis device 1 illustrated in FIG. 1 includes a log acquiring unit 11, a nursing care data acquiring unit 12, a presymptomatic disease diagnosing unit 13, and a display processing unit 14.

The log acquiring unit 11 is implemented by, for example, a log acquiring circuit 21 illustrated in FIG. 2.

The log acquiring unit 11 acquires a log indicating a change in the body of a person to be diagnosed for a presymptomatic disease.

Here, the log acquiring unit 11 acquires a log indicating a change in the body. However, this is merely an example, and the log acquiring unit 11 may acquire a log indicating an operation history of a device by the person to be diagnosed instead of the log indicating the change in the body.

In addition, the log acquiring unit 11 may acquire both a log indicating a change in the body and a log indicating an operation history of the device by the person to be diagnosed.

The log acquiring unit 11 outputs the log to the presymptomatic disease diagnosing unit 13.

The nursing care data acquiring unit 12 is implemented by, for example, a nursing care data acquiring circuit 22 illustrated in FIG. 2.

The nursing care data acquiring unit 12 acquires nursing care data indicating a nursing care content for a person to be diagnosed.

The nursing care data acquiring unit 12 outputs the nursing care data to the presymptomatic disease diagnosing unit 13.

The presymptomatic disease diagnosing unit 13 is implemented by, for example, a presymptomatic disease diagnosing circuit 23 illustrated in FIG. 2.

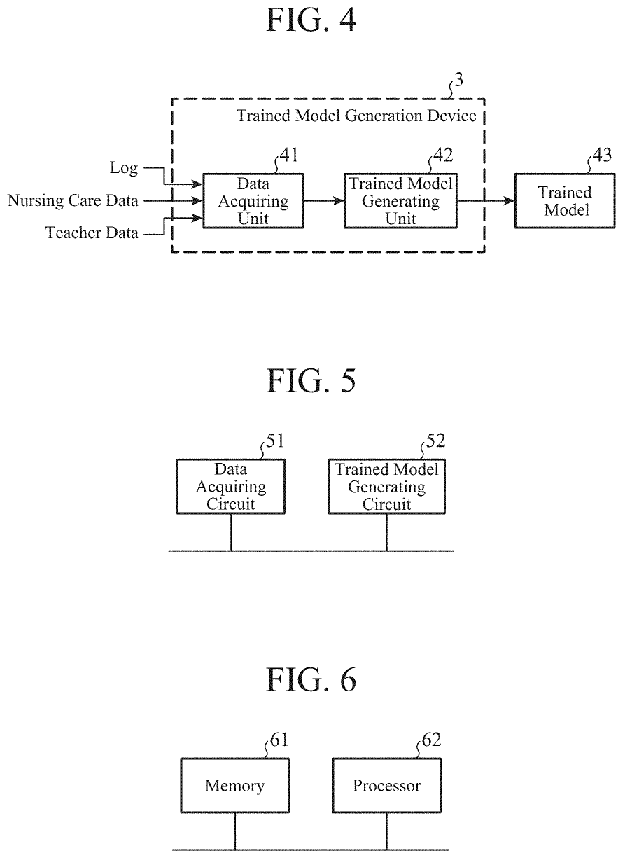

The presymptomatic disease diagnosing unit 13 includes a trained model 43 generated by the trained model generation device 3 illustrated in FIG. 4.

The presymptomatic disease diagnosing unit 13 gives the log acquired by the log acquiring unit 11 and the nursing care data acquired by the nursing care data acquiring unit 12 to the trained model 43, and acquires diagnostic data indicating a presymptomatic disease possibly occurring in the person to be diagnosed from the trained model 43.

The presymptomatic disease diagnosing unit 13 outputs the diagnostic data to the display processing unit 14.

The diagnostic data output from the presymptomatic disease diagnosing unit 13 to the display processing unit 14 includes data indicating presymptomatic disease in the abnormal finding absent state among presymptomatic diseases possibly occurring in the person to be diagnosed. The diagnostic data may include data indicating presymptomatic disease in the abnormal finding present state.

The display processing unit 14 is implemented by, for example, a display processing circuit 24 illustrated in FIG. 2.

The display processing unit 14 generates display data for displaying information on a presymptomatic disease possibly occurring in the person to be diagnosed on a screen according to the diagnostic data output from the presymptomatic disease diagnosing unit 13.

The display processing unit 14 outputs the display data to the display device 2.

The display device 2 is implemented by, for example, a liquid crystal display.

The display device 2 displays information on a presymptomatic disease possibly occurring in the person to be diagnosed on the screen according to the display data output from the display processing unit 14.

In FIG. 1, it is assumed that each of the log acquiring unit 11, the nursing care data acquiring unit 12, the presymptomatic disease diagnosing unit 13, and the display processing unit 14, which are components of the presymptomatic disease diagnosis device 1, is implemented by dedicated hardware as illustrated in FIG. 2. That is, it is assumed that the presymptomatic disease diagnosis device 1 is implemented by the log acquiring circuit 21, the nursing care data acquiring circuit 22, the presymptomatic disease diagnosing circuit 23, and the display processing circuit 24.

Each of the log acquiring circuit 21, the nursing care data acquiring circuit 22, the presymptomatic disease diagnosing circuit 23, and the display processing circuit 24 corresponds to, for example, a single circuit, a composite circuit, a programmed processor, a parallel-programmed processor, an application specific integrated circuit (ASIC), a field-programmable gate array (FPGA), or a combination thereof.

The components of the presymptomatic disease diagnosis device 1 are not limited to those implemented by dedicated hardware, and the presymptomatic disease diagnosis device 1 may be implemented by software, firmware, or a combination of software and firmware.

The software or firmware is stored in a memory of a computer as a program. The computer means hardware that executes a program, and corresponds to, for example, a central processing unit (CPU), a central processing unit, a processing unit, an arithmetic unit, a microprocessor, a microcomputer, a processor, or a digital signal processor (DSP).

FIG. 3 is a hardware configuration diagram of a computer in a case where the presymptomatic disease diagnosis device 1 is implemented by software, firmware, or the like.

In a case where the presymptomatic disease diagnosis device 1 is implemented by software, firmware, or the like, a program for causing a computer to execute each processing procedure in the log acquiring unit 11, the nursing care data acquiring unit 12, the presymptomatic disease diagnosing unit 13, and the display processing unit 14 is stored in a memory 31. Then, a processor 32 of the computer executes the program stored in the memory 31.

In addition, FIG. 2 illustrates an example in which each of the components of the presymptomatic disease diagnosis device 1 is implemented by dedicated hardware, and FIG. 3 illustrates an example in which the presymptomatic disease diagnosis device 1 is implemented by software, firmware, or the like. However, this is merely an example, and some of the components in the presymptomatic disease diagnosis device 1 may be implemented by dedicated hardware, and the remaining components may be implemented by software, firmware, or the like.

FIG. 4 is a configuration diagram illustrating the trained model generation device 3 according to the first embodiment.

FIG. 5 is a hardware configuration diagram illustrating hardware of the trained model generation device 3 according to the first embodiment.

The trained model generation device 3 illustrated in FIG. 4 includes a data acquiring unit 41 and a trained model generating unit 42.

The data acquiring unit 41 is implemented by, for example, a data acquiring circuit 51 illustrated in FIG. 5.

The data acquiring unit 41 acquires a log indicating a change in the body of the person to be diagnosed for a presymptomatic disease.

Here, the data acquiring unit 41 acquires a log indicating a change in the body. However, this is merely an example, and the data acquiring unit 41 may acquire a log indicating an operation history of a device by the person to be diagnosed for a presymptomatic disease instead of the log indicating the change in the body.

In addition, the data acquiring unit 41 may acquire both a log indicating a change in the body and a log indicating an operation history of the device by the person to be diagnosed.

In addition, the data acquiring unit 41 acquires nursing care data indicating a nursing care content for the person to be diagnosed.

Further, the data acquiring unit 41 acquires teacher data indicating presymptomatic disease possibly occurring in the person to be diagnosed or teacher data indicating not presymptomatic disease. It is assumed that the teacher data is generated by a doctor or the like.

The data acquiring unit 41 outputs each of the log, the nursing care data, and the teacher data to the trained model generating unit 42.

The trained model generating unit 42 is implemented by, for example, a trained model generating circuit 52 illustrated in FIG. 5.

The trained model generating unit 42 acquires each of the log, the nursing care data, and the teacher data from the data acquiring unit 41.

The trained model generating unit 42 uses each of the log, the nursing care data, and the teacher data to learn a presymptomatic disease possibly occurring in the person to be diagnosed, and generates the trained model 43 that outputs diagnostic data indicating the presymptomatic disease possibly occurring in the person to be diagnosed when a log indicating a change in the body of the person to be diagnosed for a presymptomatic disease and nursing care data indicating a nursing care content for the person to be diagnosed for a presymptomatic disease are given.

The trained model generating unit 42 provides the generated learned trained model 43 to the presymptomatic disease diagnosing unit 13 of the presymptomatic disease diagnosis device 1 illustrated in FIG. 1.

The learned trained model 43 learns a presymptomatic disease possibly occurring in the person to be diagnosed using each of the log, the nursing care data, and the teacher data, and is implemented by, for example, a neural network.

In FIG. 4, it is assumed that each of the data acquiring unit 41 and the trained model generating unit 42, which are components of the trained model generation device 3, is implemented by dedicated hardware as illustrated in FIG. 5. That is, it is assumed that the trained model generation device 3 is implemented by the data acquiring circuit 51 and the trained model generating circuit 52.

Each of the data acquiring circuit 51 and the trained model generating circuit 52 corresponds to, for example, a single circuit, a composite circuit, a programmed processor, a parallel-programmed processor, ASIC, FPGA, or a combination thereof.

The components of the trained model generation device 3 are not limited to those implemented by dedicated hardware, and the trained model generation device 3 may be implemented by software, firmware, or a combination of software and firmware.

FIG. 6 is a hardware configuration diagram of a computer in a case where the trained model generation device 3 is implemented by software, firmware, or the like.

In a case where the trained model generation device 3 is implemented by software, firmware, or the like, a program for causing a computer to execute each processing procedure in the data acquiring unit 41 and the trained model generating unit 42 is stored in a memory 61. Then, a processor 62 of the computer executes the program stored in the memory 61.

In addition, FIG. 5 illustrates an example in which each of the components of the trained model generation device 3 is implemented by dedicated hardware, and FIG. 6 illustrates an example in which the trained model generation device 3 is implemented by software, firmware, or the like. However, this is merely an example, and some components in the trained model generation device 3 may be implemented by dedicated hardware, and the remaining components may be implemented by software, firmware, or the like.

Next, the operation of the presymptomatic disease diagnosis device 1 illustrated in FIG. 1 will be described.

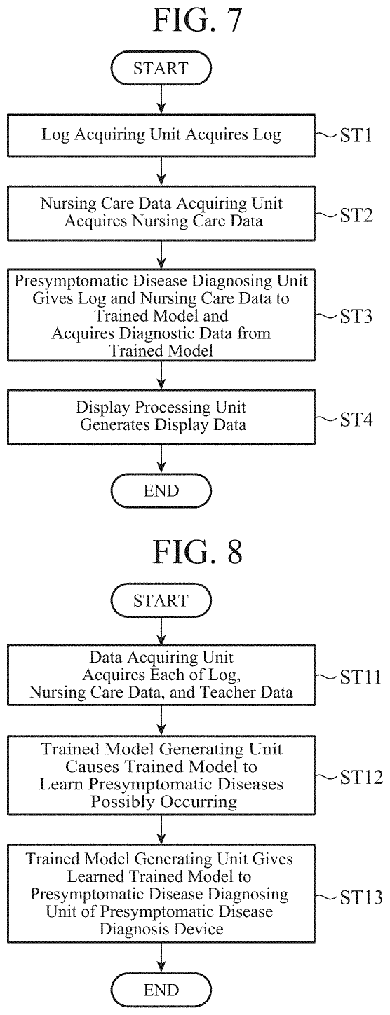

FIG. 7 is a flowchart illustrating a presymptomatic disease diagnosis method which is a processing procedure of the presymptomatic disease diagnosis device 1 illustrated in FIG. 1.

The log acquiring unit 11 acquires a log indicating a change in the body of the person to be diagnosed for a presymptomatic disease (step ST1 in FIG. 7).

In addition, the log acquiring unit 11 acquires a log indicating an operation history of the device by the person to be diagnosed for a presymptomatic disease.

The log acquiring unit 11 outputs the acquired log to the presymptomatic disease diagnosing unit 13.

If the log indicating the change in the body of the person to be diagnosed is, for example, a sleep log indicating the change in the sleeping state of the person to be diagnosed, the log acquiring unit 11 can acquire the log from an electroencephalogram analysis device that analyzes the electroencephalogram of the person to be diagnosed, an electroencephalogram sensor attached to the person to be diagnosed, or the like. Since the electroencephalogram analysis device itself is a known device, a detailed description thereof will be omitted.

If the log indicating the change in the body of the person to be diagnosed is, for example, image data indicating a state change during a meal in the person to be diagnosed, the log acquiring unit 11 can acquire the log from a video camera or the like that is photographing the person to be diagnosed.

If the log indicating the change in the body of the person to be diagnosed is, for example, a walking log indicating a change in a walking state of the person to be diagnosed, the log acquiring unit 11 can acquire the log from a walking analysis device or the like that analyzes walking of the person to be diagnosed. Since the walking analysis device itself is a known device, detailed description thereof will be omitted.

When the log acquired by the log acquiring unit 11 is, for example, an operation log indicating an operation history of a device by the person to be diagnosed, the log acquiring unit 11 can acquire the log from the device operated by the person to be diagnosed. The device operated by the person to be diagnosed is an Internet of Things (IoT) device such as an air conditioner or a television.

The nursing care data acquiring unit 12 acquires nursing care data from, for example, a nursing care recording device (not illustrated) or the like that records nursing care data indicating a nursing care content for the person to be diagnosed (step ST2 in FIG. 7).

The nursing care data acquiring unit 12 outputs the nursing care data to the presymptomatic disease diagnosing unit 13.

The presymptomatic disease diagnosing unit 13 gives the log acquired by the log acquiring unit 11 and the nursing care data acquired by the nursing care data acquiring unit 12 to the trained model 43, and acquires diagnostic data indicating a presymptomatic disease possibly occurring in the person to be diagnosed from the trained model 43 (step ST3 in FIG. 7).

The presymptomatic disease diagnosing unit 13 outputs the diagnostic data to the display processing unit 14.

The relationship among the change in the body indicated by the log or the operation history of the device indicated by the log, the nursing care content for the person to be diagnosed indicated by the nursing care data, and the presymptomatic disease possibly occurring indicated by the diagnostic data will be exemplified below.

(1) In a Case where the Presymptomatic Disease is in the Pre-Stage State of Insomnia
(a) The log is a sleep log indicating a change in a sleeping state of the person to be diagnosed. The sleeping state is classified into REM sleep, non-REM sleep, or the like. The sleep log includes data indicating a time of the sleeping state of each sleep.
(b) In the nursing care data, in addition to whether or not the person to be diagnosed takes the sleeping medication, the amount of exercise of the person to be diagnosed and the like are recorded.
(c) When it is recorded in the nursing care data that the sleeping medication is taken, the sleep log indicates that the sleeping state is normal. However, when it is not recorded in the nursing care data that the sleeping medication is taken or when it is recorded in the nursing care data that the sleeping medication is not taken, the sleep log does not indicate that the sleeping state is obviously abnormal, but indicates that the REM sleep time or the non-REM sleep time is shorter than when the sleeping state is normal. In such a case, since the person to be diagnosed sleeps due to the efficacy of the sleeping medication, and there is a possibility that sufficient sleep cannot be obtained unless the sleeping medication is taken, the trained model 43 outputs diagnostic data indicating a pre-stage state of insomnia as a presymptomatic disease in the abnormal finding absent state.
(d) In the past, when it is recorded in the nursing care data that the sleeping medication is taken, the sleep log indicates that the sleeping state is normal. Recently, the sleep log indicates that the sleeping state is abnormal even when it is recorded in the nursing care data that the sleeping medication is taken. In such a case, the trained model 43 outputs diagnostic data indicating a pre-stage state of insomnia as a presymptomatic disease in the abnormal finding present state.
(e) When it is recorded in the nursing care data that the amount of exercise of the person to be diagnosed is sufficient, the sleep log indicates that the sleeping state is normal. However, when it is recorded in the nursing care data that the amount of exercise of the person to be diagnosed is not sufficient, the sleep log does not indicate that the sleeping state is obviously abnormal, but indicates that the REM sleep time or the non-REM sleep time is reduced as compared with the case where the sleeping state is normal. In such a case, it is highly likely that insomnia is not pathological because the person to be diagnosed cannot sleep only due to lack of exercise but can sleep if exercise is sufficiently performed. Therefore, in such a case, the trained model 43 outputs diagnostic data indicating that it is not the pre-stage state of insomnia. The sufficient amount of exercise is determined according to the age or the like of the person to be diagnosed.

(2) In a Case where the Presymptomatic Disease is in the Pre-Stage State of Aspiration Pneumonia
(a) The log is image data indicating a state change during a meal in the person to be diagnosed. The image data is data including audio data. In the image data, the posture of the person to be diagnosed during a meal is shown, and a coughing sound during a meal may be recorded.
(b) The meal content of the person to be diagnosed is recorded in the nursing care data. The meal content includes cooking ingredients in addition to the menu.
(c) When a remarkable symptom considered to be a symptom of aspiration pneumonia is not recorded in the nursing care data, the image data indicates that a posture during a meal is a posture unsuitable for a meal, and a coughing sound during a meal is recorded in the image data. In such a case, since there is a high possibility of developing aspiration pneumonia, the trained model 43 outputs diagnostic data indicating a pre-stage state of aspiration pneumonia as a presymptomatic disease in the abnormal finding absent state. The posture unsuitable for a meal is, for example, an upward posture in which the jaw is raised (during backward bending).
(d) When a remarkable symptom considered to be a symptom of aspiration pneumonia is not recorded in the nursing care data, the image data indicates that the posture during a meal is a posture unsuitable for a meal, and it is recorded in the nursing care data that the meal content is a meal content that is likely to cause aspiration pneumonia.

However, if the coughing sound during the meal is not recorded in the image data and the fact that the user may cough during the meal is not recorded in the nursing care data, there is a high possibility that the function related to swallowing is not deteriorated. Therefore, the trained model 43 outputs the diagnostic data indicating that it is not the pre-stage state of aspiration pneumonia. The meal content that is likely to cause aspiration pneumonia is, for example, food that is dry with less moisture, such as bread or potato, and food that is likely to stick to the throat, such as baked layer or wakame.

(3) In a Case where the Presymptomatic Disease is in the Pre-Stage State of a Walking Disorder
(a) The log is a walking log indicating a change in a walking state of the person to be diagnosed. The walking log includes skeleton data indicating a change in the skeleton of the person to be diagnosed in addition to data indicating a walking speed, a posture, a leg raising length, a stride length, and the like of the person to be diagnosed.
(b) In the nursing care data, in addition to the walking amount of the person to be diagnosed, whether or not the person to be diagnosed takes the sleeping medication, the complexion, the conversation amount, the meal amount, or the like of the person to be diagnosed is recorded.
(c) When a remarkable symptom considered to be a walking disorder is not recorded in the nursing care data, the walking log does not indicate that the walking state is obviously abnormal, but indicates that the walking state is deteriorated. For example, when the walking speed five days before is slower than the walking speed ten days before and the walking speed today is slower than the walking speed five days before, or when the walking width five days before is narrower than the walking width ten days before and the walking width today is narrower than the walking width five days before, it is considered that the walking state is deteriorated.

At this time, if the walking amount of the person to be diagnosed recorded in the nursing care data does not exceed the walking amount in which overwork is assumed, it is considered that the cause of deterioration of the walking state is not walking fatigue. In addition, the complexion of the person to be diagnosed is good, and the meal amount is normal. In such a case, there is a high possibility that the walking state is deteriorated due to the deterioration in the walking function of the person to be diagnosed, and thus, the trained model 43 outputs the diagnostic data indicating the pre-stage state of the walking disorder as a presymptomatic disease in the abnormal finding absent state.
(d) In the past, no remarkable symptom considered as a walking disorder is recorded in the nursing care data, and the walking log does not indicate that the walking state is obviously abnormal. Recently, a remarkable symptom considered to be a walking disorder is recorded in the nursing care data. Alternatively, the walking log indicates that the walking state is obviously abnormal. In such a case, the trained model 43 outputs diagnostic data indicating a pre-stage state of the walking disorder as a presymptomatic disease in the abnormal finding present state.
(e) When a remarkable symptom considered as a walking disorder is not recorded in the nursing care data, the walking log does not indicate that the walking state is obviously abnormal, but indicates that the walking state is deteriorated. However, when the walking amount of the person to be diagnosed recorded in the nursing care data exceeds the walking amount for which overwork is assumed, it is conceivable that the cause of deterioration of the walking state is walking fatigue. In addition, when the complexion of the person to be diagnosed is bad, the conversation amount is extremely small, or the meal amount is extremely small, it is conceivable that the physical condition of the person to be diagnosed is bad. In such a case, since there is a high possibility that the walking state is deteriorated due to the cause other than the deterioration in the walking function of the person to be diagnosed, the trained model 43 outputs the diagnostic data indicating that it is not the pre-stage state of the walking disorder.

(4) In a Case where the Presymptomatic Disease is in a Pre-Stage State of Dementia
(a) The log is an operation log indicating an operation history of the device by the person to be diagnosed. The operation log is an operation history of an air conditioner, an operation history of a television, or the like.
(b) In the nursing care data, erroneous operation or the like of the device by the person to be diagnosed is recorded. Examples of the erroneous operation of the device correspond to an operation of operating the air conditioner in the heating mode when the current room temperature is a high temperature of, for example, 30 degrees or more, and an operation of operating the air conditioner in the cooling mode when the current room temperature is a low temperature of, for example, 10 degrees or less. In addition, examples of the erroneous operation of the device correspond to an operation of adjusting a channel of the television to a non-broadcast channel, and an operation of setting the volume of the television to the maximum volume.

In the presymptomatic disease diagnosis device 1 illustrated in FIG. 1, it is assumed that a staff or the like who cares for the person to be diagnosed records an erroneous operation of the device by the person to be diagnosed on the basis of the operation history of the device indicated by the operation log. That is, it is assumed that an erroneous operation of the device by the person to be diagnosed is recorded in the nursing care data. Alternatively, it is assumed that the operation log includes data indicating an erroneous operation of the device by the person to be diagnosed.
(c) The nursing care data does not record a remarkable symptom considered to be a symptom of dementia. However, if the operation log or the nursing care data indicates that the same erroneous operation is repeated a predetermined number of times even if the frequency of the erroneous operation of the device is low, the trained model 43 outputs the diagnostic data indicating that it is the pre-stage state of dementia as the presymptomatic disease in the abnormal finding absent state.
(d) The nursing care data does not record a remarkable symptom considered to be a symptom of dementia. However, if the operation log or the nursing care data indicates that the same erroneous operation is not repeated even if the frequency of the erroneous operation of the device is high, there is a high possibility that the erroneous operation is irrelevant to dementia. Therefore, in such a case, the trained model 43 outputs diagnostic data indicating that it is not the pre-stage state of dementia.

However, if the operation log or the nursing care data indicates that the frequency of the erroneous operation of the device is extremely high, even if the same erroneous operation is not repeated, the trained model 43 outputs the diagnostic data indicating that it is a pre-stage state of dementia as the presymptomatic disease in the abnormal finding absent state.

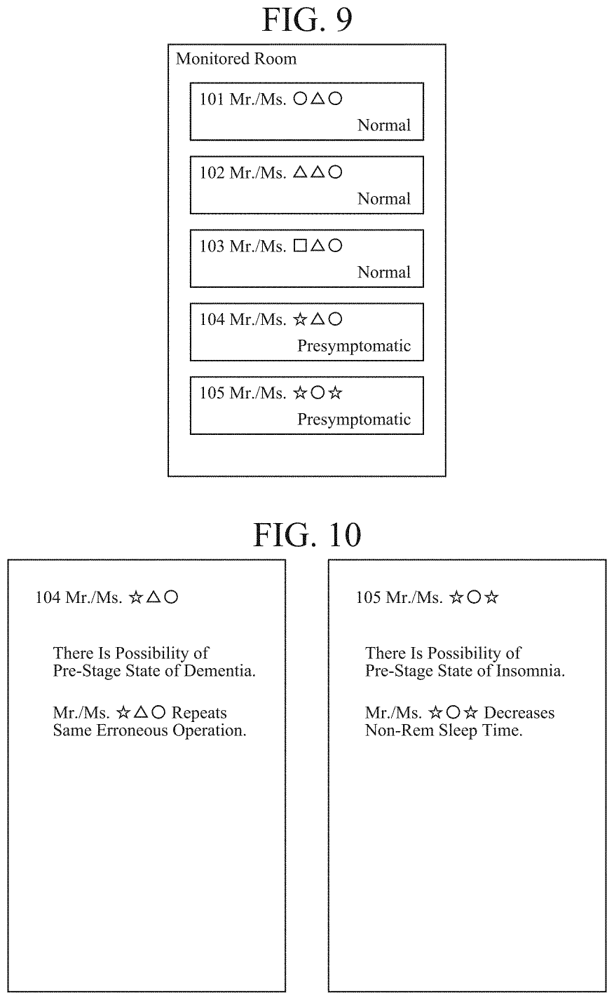

As illustrated in FIG. 9, the display processing unit 14 generates display data for displaying information indicating a diagnostic result of a presymptomatic disease for each person to be diagnosed on the screen according to the diagnostic data output from the presymptomatic disease diagnosing unit 13.

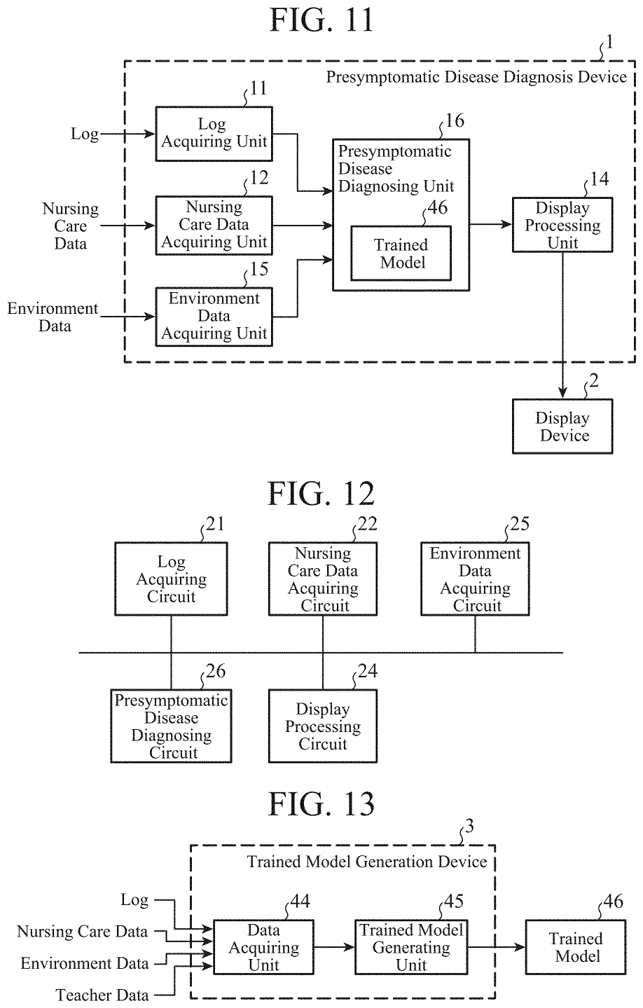

In addition, as illustrated in FIG. 10, the display processing unit 14 generates display data for displaying information on a presymptomatic disease possibly occurring in each person to be diagnosed on the screen according to the diagnostic data output from the presymptomatic disease diagnosing unit 13 (step ST4 in FIG. 7).

The display processing unit 14 generates display data for displaying the pre-stage state of insomnia on the screen when the person to be diagnosed is in the pre-stage state of insomnia, for example, and generates display data for displaying the pre-stage state of a walking disorder on the screen when the person to be diagnosed is in the pre-stage state of the walking disorder, for example.

In addition, the display processing unit 14 generates display data for displaying the pre-stage state of dementia on the screen when the person to be diagnosed is in the pre-stage state of dementia, for example, and generates display data for displaying the pre-stage state of aspiration pneumonia on the screen when the person to be diagnosed is in the pre-stage state of aspiration pneumonia, for example.

The display processing unit 14 outputs the display data to the display device 2. The display device 2 displays information on a presymptomatic disease possibly occurring in the person to be diagnosed on the screen according to the display data output from the display processing unit 14.

FIG. 9 is an explanatory diagram illustrating a diagnostic result of a presymptomatic disease for the person to be diagnosed.

In the example of FIG. 9, it is indicated that among the plurality of persons to be diagnosed, “Mr. ◯Δ◯” in room No. 101, “Mr. ΔΔ◯” in room No. 102, and “Mr. □Δ◯” in room No. 103 have no presymptomatic disease and are normal.

On the other hand, it is indicated that “Mr. ⋆Δ◯” in room 104 and “Mr. ⋆◯⋆” in room 105 have presymptomatic disease.

FIG. 10 is an explanatory diagram illustrating information on a presymptomatic disease possibly occurring in the person to be diagnosed.

The example of FIG. 10 indicates that “Mr. ⋆Δ◯” in room 104 has a possibility of being in a pre-stage state of dementia as a presymptomatic disease.

In addition, the example of FIG. 10 indicates that “Mr. -AO” in room 105 has a possibility of being in a pre-stage state of insomnia as a presymptomatic disease.

In the first embodiment described above, the presymptomatic disease diagnosis device 1 is configured to include: the log acquiring unit 11 to acquire a log indicating a change in a body of a person to be diagnosed for a presymptomatic disease; the nursing care data acquiring unit 12 to acquire nursing care data indicating a nursing care content for the person to be diagnosed; and the presymptomatic disease diagnosing unit 13 to give the log acquired by the log acquiring unit 11 and the nursing care data acquired by the nursing care data acquiring unit 12 to a trained model 43 and acquire, from the trained model 43, diagnostic data indicating presymptomatic disease possibly occurring in the person to be diagnosed. Therefore, the presymptomatic disease diagnosis device 1 can diagnose a presymptomatic disease in an abnormal finding absent state.

Next, the operation of the trained model generation device 3 illustrated in FIG. 4 will be described.

FIG. 8 is a flowchart illustrating a trained model generation method which is a processing procedure of the trained model generation device 3 illustrated in FIG. 4.

The data acquiring unit 41 acquires a log indicating a change in the body of the person to be diagnosed for a presymptomatic disease (step ST11 in FIG. 8).

In addition, the data acquiring unit 41 acquires a log indicating an operation history of a device by the person to be diagnosed for a presymptomatic disease.

Furthermore, the data acquiring unit 41 acquires nursing care data indicating a nursing care content for the person to be diagnosed, and acquires teacher data indicating presymptomatic disease possibly occurring in the person to be diagnosed or teacher data indicating not presymptomatic disease (step ST11 in FIG. 8).

The data acquiring unit 41 outputs each of the log, the nursing care data, and the teacher data to the trained model generating unit 42.

If the log is, for example, a sleep log indicating a change in the sleeping state of the person to be diagnosed, the data acquiring unit 41 can acquire the log from an electroencephalogram analysis device that analyzes the electroencephalogram of the person to be diagnosed, an electroencephalogram sensor attached to the person to be diagnosed, or the like.

If the log is, for example, image data indicating a state change during a meal in the person to be diagnosed, the data acquiring unit 41 can acquire the log from a video camera or the like that photographs the person to be diagnosed.

If the log is, for example, a walking log indicating a change in the walking state of the person to be diagnosed, the data acquiring unit 41 can acquire the log from a walking analysis device or the like that analyzes the walking of the person to be diagnosed.

If the log is, for example, an operation log indicating an operation history of the device by the person to be diagnosed, the data acquiring unit 41 can acquire the log from the device operated by the person to be diagnosed.

The data acquiring unit 41 can acquire the nursing care data from, for example, a nursing care recording device or the like that records the nursing care data indicating the nursing care content for the person to be diagnosed.

The teacher data indicates a presymptomatic disease possibly occurring in the person to be diagnosed or not a presymptomatic disease, and is assumed to be generated by a doctor or the like.

The trained model generating unit 42 acquires each of the log, the nursing care data, and the teacher data from the data acquiring unit 41.

The trained model generating unit 42 causes the trained model 43 to learn a presymptomatic disease possibly occurring in the person to be diagnosed using each of the log, the nursing care data, and the teacher data (step ST12 in FIG. 8).

Learning by the trained model 43 will be exemplified below.

(1) The nursing care data in which it is not recorded that the sleeping medication is taken or the nursing care data in which it is recorded that the sleeping medication is not taken is acquired by the trained model generating unit 42.

In addition, a sleep log not indicating that the sleeping state is clearly abnormal but indicating that the REM sleep time or the non-REM sleep time is shorter than when the sleeping state is normal is acquired by the trained model generating unit 42.

In addition, teacher data indicating a pre-stage state of insomnia is acquired by the trained model generating unit 42.

In a case where the sleep log, the nursing care data, and the teacher data as described above are acquired, the trained model generating unit 42 causes the trained model 43 to perform learning so that diagnostic data indicating a pre-stage state of insomnia is output from the trained model 43 as a presymptomatic disease in the abnormal finding absent state. In a case where the trained model 43 is implemented by the neural network, the trained model generating unit 42 changes the connection strength of the synapse of the neural network so that diagnostic data indicating a pre-stage state of insomnia is output from the trained model 43.

(2) In the nursing care data previously acquired by the trained model generating unit 42, it is recorded that the sleeping medicine is taken, and the sleep log previously acquired by the trained model generating unit 42 indicates that the sleeping state is normal.

In the nursing care data recently acquired by the trained model generating unit 42, it is not recorded that the sleeping medication is taken, or it is recorded that the sleeping medication is not taken. The sleep log recently acquired by the trained model generating unit 42 indicates that the sleeping state is abnormal.

The teaching data indicating a pre-stage state of insomnia is acquired by the trained model generating unit 42.

In a case where the sleep log, the nursing care data, and the teacher data as described above are acquired, the trained model generating unit 42 causes the trained model 43 to perform learning so that diagnostic data indicating a pre-stage state of insomnia is output from the trained model 43 as a presymptomatic disease in the abnormal finding present state. In a case where the trained model 43 is implemented by the neural network, the trained model generating unit 42 changes the connection strength of the synapse of the neural network so that diagnostic data indicating a pre-stage state of insomnia is output from the trained model 43.

(3) The nursing care data in which it is recorded that the amount of exercise of the person to be diagnosed is sufficient is acquired by the trained model generating unit 42, and the sleep log indicating that the sleeping state is normal is acquired by the trained model generating unit 42.

In addition, the nursing care data in which it is recorded that the amount of exercise of the person to be diagnosed is not sufficient is acquired by the trained model generating unit 42, and a sleep log not indicating that the sleeping state is obviously abnormal, but indicating that the REM sleep time or the non-REM sleep time is shorter than when the sleeping state is normal is acquired by the trained model generating unit 42.

In addition, the teacher data indicating that it is not a pre-stage state of insomnia is acquired by the trained model generating unit 42.

In a case where the sleep log, the nursing care data, and the teacher data as described above are acquired, the trained model generating unit 42 causes the trained model 43 to perform learning so that diagnostic data indicating that it is not the pre-stage state of insomnia is output from the trained model 43. In a case where the trained model 43 is implemented by the neural network, the trained model generating unit 42 changes the connection strength of the synapse of the neural network so that diagnostic data indicating that it is not the pre-stage state of insomnia is output from the trained model 43.

(4) The nursing care data in which no remarkable symptom considered to be a symptom of aspiration pneumonia is recorded is acquired by the trained model generating unit 42.

In addition, a log, which is image data indicating that a posture during a meal is a posture unsuitable for a meal and recording a coughing sound during the meal, is acquired by the trained model generating unit 42.

In addition, the teacher data indicating a pre-stage state of aspiration pneumonia is acquired by the trained model generating unit 42.

In a case where the image data, the nursing care data, and the teacher data as described above are acquired, the trained model generating unit 42 causes the trained model 43 to perform learning so that the diagnostic data indicating the pre-stage state of aspiration pneumonia is output from the trained model 43 as a presymptomatic disease in the abnormal finding absent state. In a case where the trained model 43 is implemented by the neural network, the trained model generating unit 42 changes the connection strength of the synapse of the neural network so that diagnostic data indicating a pre-stage state of aspiration pneumonia is output from the trained model 43.

(5) The nursing care data in which no remarkable symptom considered to be a symptom of aspiration pneumonia is recorded is acquired by the trained model generating unit 42.

In addition, a log that is image data in which a coughing sound during a meal is not recorded or nursing care data in which there is no record indicating that the user may cough during a meal is acquired by the trained model generating unit 42.

In addition, teacher data indicating that it is not a pre-stage state of aspiration pneumonia is acquired by the trained model generating unit 42.

In a case where the image data, the nursing care data, and the teacher data as described above are acquired, the trained model generating unit 42 causes the trained model 43 to perform learning so that diagnostic data indicating that it is not the pre-stage state of aspiration pneumonia is output from the trained model 43. In a case where the trained model 43 is implemented by the neural network, the trained model generating unit 42 changes the connection strength of the synapse of the neural network so that diagnostic data indicating that it is not the pre-stage state of aspiration pneumonia is output from the trained model 43.

(6) The trained model generating unit 42 acquires nursing care data in which no remarkable symptom considered to be a walking disorder is recorded but it is recorded that the walking amount of the person to be diagnosed is not an overwork walking amount.

In addition, a walking log not indicating that the walking state is obviously abnormal but indicating that the walking state is deteriorated is acquired by the trained model generating unit 42.

In addition, the teacher data indicating that it is a pre-stage state of a walking disorder is acquired by the trained model generating unit 42.

In a case where the walking log, the nursing care data, and the teacher data as described above are acquired, the trained model generating unit 42 causes the trained model 43 to perform learning so that diagnostic data indicating a pre-stage state of the walking disorder is output from the trained model 43 as a presymptomatic disease in the abnormal finding absent state. In a case where the trained model 43 is implemented by the neural network, the trained model generating unit 42 changes the connection strength of the synapse of the neural network so that diagnostic data indicating a pre-stage state of the walking disorder is output from the trained model 43.

(7) In the nursing care data previously acquired by the trained model generating unit 42, a remarkable symptom considered to be a walking disorder is not recorded. The walking log previously acquired by the trained model generating unit 42 does not indicate that the walking state is obviously abnormal.

In the nursing care data recently acquired by the trained model generating unit 42, a remarkable symptom considered to be a walking disorder is recorded. Alternatively, the walking log recently acquired by the trained model generating unit 42 indicates that the walking state is abnormal.

In addition, the teacher data indicating that it is a pre-stage state of a walking disorder is acquired by the trained model generating unit 42.

In a case where the walking log, the nursing care data, and the teacher data as described above are acquired, the trained model generating unit 42 causes the trained model 43 to perform learning so that diagnostic data indicating a pre-stage state of the walking disorder is output from the trained model 43 as a presymptomatic disease in the abnormal finding present state. In a case where the trained model 43 is implemented by the neural network, the trained model generating unit 42 changes the connection strength of the synapse of the neural network so that diagnostic data indicating a pre-stage state of the walking disorder is output from the trained model 43.

(8) The trained model generating unit 42 acquires nursing care data in which no remarkable symptom considered to be a walking disorder is recorded, but the amount of walking of the person to be diagnosed is recorded to be the amount of overwork walking.

In addition, a walking log not indicating that the walking state is obviously abnormal but indicating that the walking state is deteriorated is acquired by the trained model generating unit 42.

In addition, the trained model generating unit 42 acquires teacher data indicating that it is not the pre-stage state of the walking disorder.

In a case where the walking log, the nursing care data, and the teacher data as described above are acquired, the trained model generating unit 42 causes the trained model 43 to perform learning so that diagnostic data indicating that it is not the pre-stage state of the walking disorder is output from the trained model 43. In a case where the trained model 43 is implemented by the neural network, the trained model generating unit 42 changes the connection strength of the synapse of the neural network so that diagnostic data indicating that it is not the pre-stage state of the walking disorder is output from the trained model 43.

(9) The trained model generating unit 42 acquires nursing care data in which no remarkable symptom considered to be a symptom of dementia is recorded.

In addition, the trained model generating unit 42 acquires an operation log indicating that the same erroneous operation is repeated a predetermined number of times even when the frequency of the erroneous operation of the device is low, or nursing care data recording that the same erroneous operation is repeated a predetermined number of times even when the frequency of the erroneous operation of the device is low.

In addition, the trained model generating unit 42 acquires teacher data indicating that it is a pre-stage state of dementia.

In a case where the operation log, the nursing care data, and the teacher data as described above are acquired, the trained model generating unit 42 causes the trained model 43 to perform learning so that diagnostic data indicating that it is a pre-stage state of dementia is output from the trained model 43 as a presymptomatic disease in the abnormal finding absent state. In a case where the trained model 43 is implemented by the neural network, the trained model generating unit 42 changes the connection strength of the synapse of the neural network so that diagnostic data indicating that it is a pre-stage state of dementia is output from the trained model 43.

(10) The trained model generating unit 42 acquires nursing care data in which no remarkable symptom considered to be a symptom of dementia is recorded.

In addition, the trained model generating unit 42 acquires an operation log indicating that the same erroneous operation is not repeated even when the frequency of the erroneous operation of the device is high, or nursing care data recording that the same erroneous operation is not repeated even when the frequency of the erroneous operation of the device is high.

In addition, the trained model generating unit 42 acquires teacher data indicating that it is not the pre-stage state of dementia.

In a case where the operation log, the nursing care data, and the teacher data as described above are acquired, the trained model generating unit 42 causes the trained model 43 to perform learning so that diagnostic data indicating that it is not the pre-stage state of dementia is output from the trained model 43. In a case where the trained model 43 is implemented by the neural network, the trained model generating unit 42 changes the connection strength of the synapse of the neural network so that diagnostic data indicating that it is not the pre-stage state of dementia is output from the trained model 43.

However, the operation log or the nursing care data indicates that the frequency of erroneous operation of the device is extremely high, and the trained model generating unit 42 acquires teacher data indicating that it is a pre-stage state of dementia. In such a case, the trained model generating unit 42 causes the trained model 43 to perform learning so that diagnostic data indicating that it is a pre-stage state of dementia is output from the trained model 43 as a presymptomatic disease in the abnormal finding absent state.

The trained model generating unit 42 provides the learned trained model 43 to the presymptomatic disease diagnosing unit 13 of the presymptomatic disease diagnosis device 1 illustrated in FIG. 1 (step ST13 in FIG. 8).

In the first embodiment described above, the trained model generation device 3 is configured to include: the data acquiring unit 41 to acquire a log indicating a change in a body of a person to be diagnosed for a presymptomatic disease, acquire nursing care data indicating a nursing care content for the person to be diagnosed, and acquire teacher data indicating a presymptomatic disease possibly occurring in the person to be diagnosed or teacher data indicating not a presymptomatic disease; and the trained model generating unit 42 to learn the presymptomatic disease possibly occurring in the person to be diagnosed by using each of the log, the nursing care data, and the teacher data acquired by the data acquiring unit 41, and generate a trained model 43 that outputs diagnostic data indicating the presymptomatic disease possibly occurring in the person to be diagnosed when the log indicating a change in the body of the person to be diagnosed for a presymptomatic disease and the nursing care data indicating a nursing care content for the person to be diagnosed for a presymptomatic disease are given. Therefore, the trained model generation device 3 can provide the trained model 43 to the presymptomatic disease diagnosis device 1 for diagnosing the presymptomatic disease in the abnormal finding absent state.

### Second Embodiment

In the second embodiment, the presymptomatic disease diagnosis device 1 in which the presymptomatic disease diagnosing unit 16 gives environment data indicating the environment around the person to be diagnosed to a trained model 46 in addition to the log and the nursing care data, and acquires diagnostic data indicating a presymptomatic disease possibly occurring in the person to be diagnosed from the trained model 46 will be described.

FIG. 11 is a configuration diagram illustrating a presymptomatic disease diagnosis device 1 according to a second embodiment.

FIG. 12 is a hardware configuration diagram illustrating hardware of the presymptomatic disease diagnosis device 1 according to the second embodiment. In FIGS. 11 and 12, the same reference numerals as those in FIGS. 1 and 2 denote the same or corresponding parts, and thus description thereof is omitted.

The presymptomatic disease diagnosis device 1 illustrated in FIG. 11 includes a log acquiring unit 11, a nursing care data acquiring unit 12, an environment data acquiring unit 15, a presymptomatic disease diagnosing unit 16, and a display processing unit 14.

The environment data acquiring unit 15 is implemented by, for example, an environment data acquiring circuit 25 illustrated in FIG. 12.

The environment data acquiring unit 15 acquires environment data indicating the environment around the person to be diagnosed.

The environment data acquiring unit 15 outputs the environment data to the presymptomatic disease diagnosing unit 16.

The presymptomatic disease diagnosing unit 16 is implemented by, for example, a presymptomatic disease diagnosing circuit 26 illustrated in FIG. 12.

The presymptomatic disease diagnosing unit 16 includes a trained model 46 generated by the trained model generation device 3 illustrated in FIG. 13.

The presymptomatic disease diagnosing unit 16 gives the log acquired by the log acquiring unit 11, the nursing care data acquired by the nursing care data acquiring unit 12, and the environment data acquired by the environment data acquiring unit 15 to the trained model 46, and acquires diagnostic data indicating a presymptomatic disease possibly occurring in the person to be diagnosed from the trained model 46.

The presymptomatic disease diagnosing unit 16 outputs the diagnostic data to the display processing unit 14.

In FIG. 11, it is assumed that each of the log acquiring unit 11, the nursing care data acquiring unit 12, the environment data acquiring unit 15, the presymptomatic disease diagnosing unit 16, and the display processing unit 14, which are components of the presymptomatic disease diagnosis device 1, is implemented by dedicated hardware as illustrated in FIG. 12. That is, it is assumed that the presymptomatic disease diagnosis device 1 is implemented by the log acquiring circuit 21, the nursing care data acquiring circuit 22, the environment data acquiring circuit 25, the presymptomatic disease diagnosing circuit 26, and the display processing circuit 24.

Each of the log acquiring circuit 21, the nursing care data acquiring circuit 22, the environment data acquiring circuit 25, the presymptomatic disease diagnosing circuit 26, and the display processing circuit 24 corresponds to, for example, a single circuit, a composite circuit, a programmed processor, a parallel-programmed processor, ASIC, FPGA, or a combination thereof.

The components of the presymptomatic disease diagnosis device 1 are not limited to those implemented by dedicated hardware, and the presymptomatic disease diagnosis device 1 may be implemented by software, firmware, or a combination of software and firmware.

In a case where the presymptomatic disease diagnosis device 1 is implemented by software, firmware, or the like, a program for causing a computer to execute each processing procedure in the log acquiring unit 11, the nursing care data acquiring unit 12, the environment data acquiring unit 15, the presymptomatic disease diagnosing unit 16, and the display processing unit 14 is stored in the memory 31 illustrated in FIG. 3. Then, the processor 32 illustrated in FIG. 3 executes the program stored in the memory 31.

In addition, FIG. 12 illustrates an example in which each of the components of the presymptomatic disease diagnosis device 1 is implemented by dedicated hardware, and FIG. 3 illustrates an example in which the presymptomatic disease diagnosis device 1 is implemented by software, firmware, or the like. However, this is merely an example, and some of the components in the presymptomatic disease diagnosis device 1 may be implemented by dedicated hardware, and the remaining components may be implemented by software, firmware, or the like.

FIG. 13 is a configuration diagram illustrating a trained model generation device 3 according to the second embodiment.

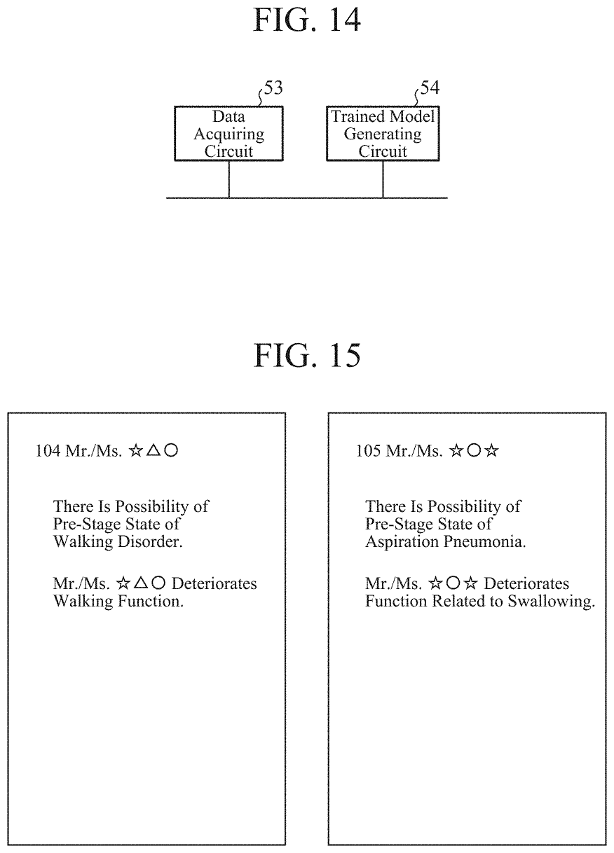

FIG. 14 is a hardware configuration diagram illustrating hardware of the trained model generation device 3 according to the second embodiment.

The trained model generation device 3 illustrated in FIG. 13 includes a data acquiring unit 44 and a trained model generating unit 45.

The data acquiring unit 44 is implemented by, for example, a data acquiring circuit 53 illustrated in FIG. 14.

The data acquiring unit 44 acquires a log indicating a change in the body of the person to be diagnosed for a presymptomatic disease.

Here, the data acquiring unit 44 acquires a log indicating a change in the body. However, this is merely an example, and the data acquiring unit 44 may acquire a log indicating an operation history of a device by the person to be diagnosed for a presymptomatic disease instead of the log indicating the change in the body.

In addition, the data acquiring unit 44 may acquire both a log indicating a change in the body and a log indicating an operation history of the device by the person to be diagnosed.

The data acquiring unit 44 acquires nursing care data indicating a nursing content for the person to be diagnosed, and acquires environment data indicating the environment around the person to be diagnosed.

In addition, the data acquiring unit 44 acquires teacher data indicating a presymptomatic disease possibly occurring in the person to be diagnosed or teacher data indicating not a presymptomatic disease. It is assumed that the teacher data is generated by a doctor or the like.

The data acquiring unit 44 outputs each of the log, the nursing care data, the environment data, and the teacher data to the trained model generating unit 45.

The trained model generating unit 45 is implemented by, for example, a trained model generating circuit 54 illustrated in FIG. 14.

The trained model generating unit 45 acquires each of the log, the nursing care data, the environment data, and the teacher data from the data acquiring unit 44.

The trained model generating unit 45 uses each of the log, the nursing care data, the environment data, and the teacher data to learn a presymptomatic disease possibly occurring in the person to be diagnosed, and generates the trained model 46 that outputs the diagnostic data indicating the presymptomatic disease possibly occurring in the person to be diagnosed when given the log indicating the change in the body of the person to be diagnosed for a presymptomatic disease, the nursing care data indicating the nursing care content for the person to be diagnosed for a presymptomatic disease, and the environment data indicating the environment around the person to be diagnosed.

The trained model generating unit 45 provides the generated learned trained model 46 to the presymptomatic disease diagnosing unit 16 of the presymptomatic disease diagnosis device 1 illustrated in FIG. 11.

The learned trained model 46 learns a presymptomatic disease possibly occurring in the person to be diagnosed using each of the log, the nursing care data, the environment data, and the teacher data, and is implemented by, for example, a neural network.

In FIG. 13, it is assumed that each of the data acquiring unit 44 and the trained model generating unit 45, which are components of the trained model generation device 3, is implemented by dedicated hardware as illustrated in FIG. 14. That is, it is assumed that the trained model generation device 3 is implemented by the data acquiring circuit 53 and the trained model generating circuit 54.

Each of the data acquiring circuit 53 and the trained model generating circuit 54 corresponds to, for example, a single circuit, a composite circuit, a programmed processor, a parallel-programmed processor, ASIC, FPGA, or a combination thereof.

The components of the trained model generation device 3 are not limited to those implemented by dedicated hardware, and the trained model generation device 3 may be implemented by software, firmware, or a combination of software and firmware.

In a case where the trained model generation device 3 is implemented by software, firmware, or the like, a program for causing a computer to execute each processing procedure in the data acquiring unit 44 and the trained model generating unit 45 is stored in the memory 61 illustrated in FIG. 6. Then, the processor 62 illustrated in FIG. 6 executes the program stored in the memory 61.

In addition, FIG. 14 illustrates an example in which each of the components of the trained model generation device 3 is implemented by dedicated hardware, and FIG. 6 illustrates an example in which the trained model generation device 3 is implemented by software, firmware, or the like. However, this is merely an example, and some components in the trained model generation device 3 may be implemented by dedicated hardware, and the remaining components may be implemented by software, firmware, or the like.

Next, the operation of the presymptomatic disease diagnosis device 1 illustrated in FIG. 11 will be described.

The log acquiring unit 11 acquires a log indicating a change in the body of a person to be diagnosed for a presymptomatic disease.

In addition, the log acquiring unit 11 acquires a log indicating an operation history of the device by the person to be diagnosed for a presymptomatic disease.

The log acquiring unit 11 outputs the acquired log to the presymptomatic disease diagnosing unit 16.

The nursing care data acquiring unit 12 acquires nursing care data from, for example, a nursing care recording device or the like that records nursing care data indicating a nursing care content for the person to be diagnosed.

The nursing care data acquiring unit 12 outputs the nursing care data to the presymptomatic disease diagnosing unit 16.

The environment data acquiring unit 15 acquires environment data indicating the environment around the person to be diagnosed.

The environment data acquiring unit 15 outputs the environment data to the presymptomatic disease diagnosing unit 16.

If the environment data is an environment log indicating, for example, room temperature, humidity, illuminance, atmospheric pressure, carbon dioxide concentration, air contamination, odor, presence or absence of an obstacle, and the like, the environment data acquiring unit 15 can acquire a log from, for example, a room temperature sensor observing room temperature, a humidity sensor observing humidity, and an illuminance sensor observing illuminance. In addition, the environment data acquiring unit 15 can acquire a log from, for example, an atmospheric pressure sensor observing atmospheric pressure, a carbon dioxide sensor observing carbon dioxide concentration, a pollution observation sensor observing air pollution, and an odor sensor observing odor. In addition, the environment data acquiring unit 15 can acquire a log from, for example, a monitoring camera photographing an environment including the person to be diagnosed. Note that the environment data includes position data indicating an installation position of a sensor observing the environment.

The presymptomatic disease diagnosing unit 16 gives the log acquired by the log acquiring unit 11, the nursing care data acquired by the nursing care data acquiring unit 12, and the environment data acquired by the environment data acquiring unit 15 to the trained model 46, and acquires diagnostic data indicating a presymptomatic disease possibly occurring in the person to be diagnosed from the trained model 46.

The presymptomatic disease diagnosing unit 16 outputs the diagnostic data to the display processing unit 14.

The relationship among the change in the body indicated by the log or the operation history of the device indicated by the log, the nursing care content for the person to be diagnosed indicated by the nursing care data, the environment indicated by the environment data, and the presymptomatic disease possibly occurring indicated by the diagnostic data will be exemplified below.

(1) In a Case where the Presymptomatic Disease is in the Pre-Stage State of Insomnia
(a) The log is a sleep log indicating a change in a sleeping state of the person to be diagnosed.
(b) In the nursing care data, in addition to whether or not the person to be diagnosed takes the sleeping medication, the amount of exercise of the person to be diagnosed and the like are recorded.
(c) The environment data includes, for example, data indicating room temperature.
(d) When it is recorded in the nursing care data that the sleeping medication is taken, the sleep log indicates that the sleeping state is normal. However, when it is not recorded in the nursing care data that the sleeping medication is taken or when it is recorded in the nursing care data that the sleeping medication is not taken, the sleep log does not indicate that the sleeping state is obviously abnormal, but indicates that the REM sleep time or the non-REM sleep time is shorter than when the sleeping state is normal. In such a case, since the person to be diagnosed sleeps due to the efficacy of the sleeping medication, and there is a possibility that sufficient sleep cannot be obtained unless the sleeping medication is taken, the trained model 46 outputs diagnostic data indicating a pre-stage state of insomnia as a presymptomatic disease in the abnormal finding absent state.

However, when the temperature indicated by the environment data is, for example, a high temperature of 30 degrees or more and the environment is an environment in which it is difficult to sleep, the sleep log indicates that the REM sleep time or the non-REM sleep time is shorter than when the sleeping state is normal. However, when the temperature indicated by the environment data is a room temperature of about 20 degrees suitable for sleep, the sleep log indicates that the sleeping state is normal. In such a case, since there is a high possibility that it is difficult to sleep due to an inappropriate sleeping environment, the trained model 46 outputs diagnostic data indicating that it is not a pre-stage state of insomnia.
(e) In the past, when it is recorded in the nursing care data that the sleeping medication is taken, the sleep log indicates that the sleeping state is normal. Recently, the sleep log indicates that the sleeping state is abnormal even when it is recorded in the nursing care data that the sleeping medication is taken. In such a case, the trained model 46 outputs diagnostic data indicating a pre-stage state of insomnia as a presymptomatic disease in the abnormal finding present state.

However, when the temperature indicated by the environment data is, for example, a high temperature of 30 degrees or more and the environment is an environment in which it is difficult to sleep, the sleep log indicates that the sleeping state is abnormal. However, when the temperature indicated by the environment data is a room temperature of about 20 degrees suitable for sleep, the sleep log indicates that the sleeping state is normal. In such a case, since there is a high possibility that it is difficult to sleep due to an inappropriate sleeping environment, the trained model 46 outputs diagnostic data indicating that it is not a pre-stage state of insomnia.
(f) When it is recorded in the nursing care data that the amount of exercise of the person to be diagnosed is sufficient, the sleep log indicates that the sleeping state is normal. However, when it is recorded in the nursing care data that the amount of exercise of the person to be diagnosed is not sufficient, the sleep log does not indicate that the sleeping state is obviously abnormal, but indicates that the REM sleep time or the non-REM sleep time is shorter than when the sleeping state is normal. In such a case, it is highly likely that insomnia is not pathological because the person to be diagnosed cannot sleep only due to lack of exercise but can sleep if exercise is sufficiently performed. Therefore, in such a case, the trained model 46 outputs diagnostic data indicating that it is not the pre-stage state of insomnia.

Note that, regardless of whether or not the amount of exercise of the person to be diagnosed is sufficient, when the room temperature is about 20 degrees suitable for sleep, the sleep log indicates that the sleeping state is normal. On the other hand, regardless of whether or not the amount of exercise of the person to be diagnosed is sufficient, when the environment is difficult to sleep, the sleep log indicates that the REM sleep time or the non-REM sleep time is shorter than when the sleeping state is normal. In such a case, since there is a high possibility that it is difficult to sleep due to an inappropriate sleeping environment, the trained model 46 outputs diagnostic data indicating that it is not a pre-stage state of insomnia.

(2) In a Case where the Presymptomatic Disease is in the Pre-Stage State of Aspiration Pneumonia
(a) The log is image data indicating a state change during a meal in the person to be diagnosed. The image data is data including audio data. In the image data, the posture of the person to be diagnosed during a meal is shown, and a coughing sound during a meal may be recorded.
(b) The meal content of the person to be diagnosed is recorded in the nursing care data. The meal content includes cooking ingredients in addition to the menu.
(c) The environment data includes, for example, data indicating the carbon dioxide concentration.
(d) No remarkable symptom considered to be a symptom of aspiration pneumonia is recorded in the nursing care data. However, the image data indicates that the posture during a meal is a posture unsuitable for a meal, and a coughing sound during a meal is recorded in the image data. In such a case, since there is a high possibility of developing aspiration pneumonia, the trained model 46 outputs diagnostic data indicating a pre-stage state of aspiration pneumonia as a presymptomatic disease in the abnormal finding absent state.

However, since the carbon dioxide concentration indicated by the environment data is higher than a reference concentration, the condition of the person to be diagnosed may be deteriorated. In such a case, since there is a high possibility that coughing occurs due to the influence of carbon dioxide, the trained model 46 outputs diagnostic data indicating that it is not a pre-stage state of aspiration pneumonia. The reference concentration is, for example, the lowest concentration at which carbon dioxide poisoning may occur.
(e) No remarkable symptom considered to be a symptom of aspiration pneumonia is recorded in the nursing care data. In addition, the image data indicates that the posture during a meal is a posture unsuitable for a meal, and the nursing care data indicates that the meal content is likely to cause aspiration pneumonia. However, when the coughing sound during a meal is not recorded in the image data or when the fact that the user may cough during a meal is not recorded in the nursing care data, since there is a high possibility that the function related to swallowing is not deteriorated, the trained model 46 outputs the diagnostic data indicating that it is not the pre-stage state of aspiration pneumonia.

(3) In a Case where the Presymptomatic Disease is in the Pre-Stage State of a Walking Disorder
(a) The log is a walking log indicating a change in a walking state of the person to be diagnosed. The walking log includes skeleton data indicating a change in the skeleton of the person to be diagnosed in addition to data indicating a walking speed, a posture, a leg raising length, a stride length, and the like of the person to be diagnosed.
(b) In the nursing care data, in addition to the walking amount of the person to be diagnosed, whether or not the person to be diagnosed takes the sleeping medication, the complexion, the conversation amount, the meal amount, or the like of the person to be diagnosed is recorded.
(c) The environment data includes, for example, video data of a monitoring camera that photographs an environment including the person to be diagnosed.
(d) No remarkable symptom considered to be a walking disorder is recorded in the nursing care data. In addition, the walking log does not indicate that the walking state is obviously abnormal, but indicates that the walking state is deteriorated.

At this time, if the walking amount of the person to be diagnosed recorded in the nursing care data does not exceed the walking amount in which overwork is assumed, it is considered that the cause of deterioration of the walking state is not walking fatigue. In addition, the complexion of the person to be diagnosed is good, and the meal amount is normal. In such a case, since there is a high possibility that the walking state is deteriorated due to the deterioration in the walking function of the person to be diagnosed, the trained model 46 outputs the diagnostic data indicating the pre-stage state of the walking disorder as a presymptomatic disease in the abnormal finding absent state.

However, if the environment data indicates that an obstacle was present during walking, it is conceivable that the walking state has deteriorated due to the influence of the obstacle. In such a case, since there is a high possibility that the walking state is deteriorated due to a cause other than the deterioration in the walking function of the person to be diagnosed, the trained model 46 outputs the diagnostic data indicating not the pre-stage state of the walking disorder.
(e) In the past, no remarkable symptom considered to be a walking disorder is recorded in the nursing care data, and the walking log does not indicate that the walking state is obviously abnormal. Recently, a remarkable symptom considered to be a walking disorder is recorded in the nursing care data. Alternatively, the walking log indicates that the walking state is obviously abnormal. In such a case, the trained model 46 outputs diagnostic data indicating a pre-stage state of the walking disorder as a presymptomatic disease in the abnormal finding present state.

However, if the environment data indicates that an obstacle was present during walking, it is conceivable that the walking state has deteriorated due to the influence of the obstacle. In such a case, since there is a high possibility that the walking state is deteriorated due to a cause other than the deterioration in the walking function of the person to be diagnosed, the trained model 46 outputs the diagnostic data indicating not the pre-stage state of the walking disorder.
(f) No remarkable symptom considered to be a walking disorder is recorded in the nursing care data. In addition, the walking log does not indicate that the walking state is obviously abnormal, but indicates that the walking state is deteriorated. However, when the walking amount of the person to be diagnosed recorded in the nursing care data exceeds the walking amount for which overwork is assumed, it is conceivable that the cause of deterioration of the walking state is walking fatigue. In addition, when the complexion of the person to be diagnosed is bad, the conversation amount is extremely small, or the meal amount is extremely small, it is conceivable that the physical condition of the person to be diagnosed is bad. In such a case, since there is a high possibility that the walking state is deteriorated due to the cause other than the deterioration in the walking function of the person to be diagnosed, the trained model 46 outputs the diagnostic data indicating that it is not the pre-stage state of the walking disorder.

(4) In a Case where the Presymptomatic Disease is in a Pre-Stage State of Dementia
(a) The log is an operation log indicating an operation history of the device by the person to be diagnosed. The operation log is an operation history of an air conditioner, an operation history of a television, or the like.
(b) In the nursing care data, erroneous operation or the like of the device by the person to be diagnosed is recorded. Examples of the erroneous operation of the device include an operation of operating the air conditioner in the heating mode when the current room temperature is a high temperature of 30 degrees or more, and an operation of operating the air conditioner in the cooling mode when the current room temperature is a low temperature of 10 degrees or less. In addition, examples of the erroneous operation of the device correspond to an operation of adjusting a channel of the television to a non-broadcast channel, and an operation of setting the volume of the television to the maximum volume.

In the presymptomatic disease diagnosis device 1 illustrated in FIG. 11, it is assumed that a staff or the like who cares for the person to be diagnosed records an erroneous operation of the device by the person to be diagnosed on the basis of the operation history of the device indicated by the operation log. That is, it is assumed that an erroneous operation of the device by the person to be diagnosed is recorded in the nursing care data. Alternatively, it is assumed that the operation log includes data indicating an erroneous operation of the device by the person to be diagnosed.
(c) The environment data includes, for example, data indicating room temperature.
(d) The nursing care data does not record a remarkable symptom considered to be a symptom of dementia. However, if the operation log or the nursing care data indicates that the same erroneous operation is repeated a predetermined number of times even if the frequency of the erroneous operation of the device is low, the trained model 46 outputs the diagnostic data indicating that it is the pre-stage state of dementia as a presymptomatic disease in the abnormal finding absent state.
(e) No remarkable symptom considered to be a symptom of dementia is recorded in the nursing care data. However, if the operation log or the nursing care data indicates that the same erroneous operation is not repeated even if the frequency of the erroneous operation of the device is high, there is a high possibility that the erroneous operation is irrelevant to dementia. Therefore, in such a case, the trained model 46 outputs diagnostic data indicating that it is not the pre-stage state of dementia.

However, if the operation log or the nursing care data indicates that the frequency of erroneous operation of the device is extremely high, the trained model 46 outputs diagnostic data indicating that it is a pre-stage state of dementia as a presymptomatic disease in the abnormal finding absent state.

In addition, when the room temperature indicated by the environment data is a dangerous temperature at which heat stroke may occur, if there is no operation log indicating an operating operation in the cooling mode of the air conditioner, there is a suspicion of dementia. Therefore, the trained model 46 outputs diagnostic data indicating that it is a pre-stage state of dementia as a presymptomatic disease in the abnormal finding absent state.

As illustrated in FIG. 9, the display processing unit 14 generates display data for displaying information indicating a diagnostic result of presymptomatic disease for each person to be diagnosed on the screen according to the diagnostic data output from the presymptomatic disease diagnosing unit 16.

In addition, as illustrated in FIG. 15, the display processing unit 14 generates display data for displaying information on a presymptomatic disease possibly occurring in each person to be diagnosed on the screen according to the diagnostic data output from presymptomatic disease diagnosing unit 16.

The display processing unit 14 outputs the display data to the display device 2. The display device 2 displays information on a presymptomatic disease possibly occurring in the person to be diagnosed on the screen according to the display data output from the display processing unit 14.

FIG. 15 is an explanatory diagram illustrating information on the presymptomatic disease possibly occurring in the person to be diagnosed.

In the example of FIG. 15, “Mr. ⋆Δ◯” in room No. 104 indicates that there is a possibility of a pre-stage state of walking disorder as a presymptomatic disease.

In addition, the example of FIG. 15 indicates that there is a possibility that “Mr. ⋆Δ⋆” in room 105 is in a pre-stage state of aspiration pneumonia as a presymptomatic disease.

In the second embodiment described above, the presymptomatic disease diagnosis device 1 illustrated in FIG. 11 is configured to include the environment data acquiring unit 15 to acquire environment data indicating an environment around the person to be diagnosed, in which the presymptomatic disease diagnosing unit 16 gives the log acquired by the log acquiring unit 11, the nursing care data acquired by the nursing care data acquiring unit 12, and the environment data acquired by the environment data acquiring unit 15 to the trained model 46, and acquires diagnostic data indicating the presymptomatic disease possibly occurring in the person to be diagnosed from the trained model 46. Therefore, the presymptomatic disease diagnosis device 1 illustrated in FIG. 11 can improve diagnosis accuracy of the presymptomatic disease as compared with the presymptomatic disease diagnosis device 1 illustrated in FIG. 1.

Next, the operation of the trained model generation device 3 illustrated in FIG. 13 will be described.

The data acquiring unit 44 acquires a log indicating a change in the body of the person to be diagnosed for a presymptomatic disease.

In addition, the data acquiring unit 44 acquires a log indicating an operation history of the device by the person to be diagnosed for a presymptomatic disease.

The data acquiring unit 44 acquires nursing care data indicating a nursing content for the person to be diagnosed, and acquires environment data indicating the environment around the person to be diagnosed.

Further, the data acquiring unit 44 acquires teacher data indicating a presymptomatic disease possibly occurring in the person to be diagnosed or teacher data indicating not presymptomatic disease.

The data acquiring unit 44 outputs each of the log, the nursing care data, the environment data, and the teacher data to the trained model generating unit 45.

The trained model generating unit 45 acquires each of the log, the nursing care data, the environment data, and the teacher data from the data acquiring unit 44.

The trained model generating unit 45 causes the trained model 46 to learn a presymptomatic disease possibly occurring in the person to be diagnosed using each of the log, the nursing care data, the environment data, and the teacher data.

Learning by the trained model 46 will be exemplified below.

(1) The nursing care data in which it is not recorded that the sleeping medication is taken or the nursing care data in which it is recorded that the sleeping medication is not taken is recorded is acquired by the trained model generating unit 45.

In addition, a sleep log not indicating that the sleeping state is obviously abnormal but indicating that the REM sleep time or the non-REM sleep time is shorter than when the sleeping state is normal is acquired by the trained model generating unit 45.

Furthermore, environment data indicating a room temperature of about 20 degrees suitable for sleep is acquired by the trained model generating unit 45.

Further, teacher data indicating a pre-stage state of insomnia is acquired by the trained model generating unit 45.

In a case where the sleep log, the nursing care data, the environment data, and the teacher data as described above are acquired, the trained model generating unit 45 causes the trained model 46 to perform learning so that diagnostic data indicating a pre-stage state of insomnia is output from the trained model 46 as a presymptomatic disease in the abnormal finding absent state. In a case where the trained model 46 is implemented by the neural network, the trained model generating unit 45 changes the connection strength of the synapse of the neural network so that diagnostic data indicating a pre-stage state of insomnia is output from the trained model 46.

(2) In the nursing care data previously acquired by the trained model generating unit 45, it is recorded that sleeping medication is taken, and the sleep log previously acquired by the trained model generating unit 45 indicates that the sleeping state is normal.

In the nursing care data recently acquired by the trained model generating unit 45, it is not recorded that the sleeping medication is taken, or it is recorded that the sleeping medication is not taken. The sleep log recently acquired by the trained model generating unit 45 indicates that the sleeping state is abnormal.

Environment data indicating a room temperature of about 20 degrees suitable for sleep is acquired by the trained model generating unit 45.

In addition, teacher data indicating a pre-stage state of insomnia is acquired by the trained model generating unit 45.

In a case where the sleep log, the nursing care data, and the teacher data as described above are acquired, the trained model generating unit 45 causes the trained model 46 to perform learning so that diagnostic data indicating a pre-stage state of insomnia is output from the trained model 46 as a presymptomatic disease in the abnormal finding present state.

(3) The nursing care data in which it is not recorded that the sleeping medication is taken or the nursing care data in which it is recorded that the sleeping medication is not taken is acquired by the trained model generating unit 45.

In addition, a sleep log not indicating that the sleeping state is obviously abnormal but indicating that the REM sleep time or the non-REM sleep time is shorter than when the sleeping state is normal is acquired by the trained model generating unit 45.

In addition, environment data indicating a room temperature of 30 degrees or more, which is an environment where it is difficult to sleep, is acquired by the trained model generating unit 45.

Further, teacher data indicating that it is not the pre-stage state of insomnia is acquired by the trained model generating unit 45.

In a case where the sleep log, the nursing care data, the environment data, and the teacher data as described above are acquired, the trained model generating unit 45 causes the trained model 46 to perform learning so that the diagnostic data indicating that it is not the pre-stage state of insomnia is output from the trained model 46.

(4) Nursing care data in which it is recorded that the amount of exercise of the person to be diagnosed is sufficient is acquired by the trained model generating unit 45, and a sleep log indicating that the sleeping state is normal is acquired by the trained model generating unit 45.

In addition, nursing care data in which it is recorded that the amount of exercise of the person to be diagnosed is not sufficient is acquired by the trained model generating unit 45, and a sleep log not indicating that the sleeping state is obviously abnormal but indicating that the REM sleep time or the non-REM sleep time is shorter than when the sleeping state is normal is acquired by the trained model generating unit 45.

Furthermore, environment data indicating the temperature of the surrounding environment is acquired by the trained model generating unit 45.

Further, teacher data indicating that it is not the pre-stage state of insomnia is acquired by the trained model generating unit 45.

In a case where the sleep log, the nursing care data, the environment data, and the teacher data as described above are acquired, the trained model generating unit 45 causes the trained model 46 to perform learning so that the diagnostic data indicating that it is not the pre-stage state of insomnia is output from the trained model 46.

(5) A sleep log indicating that the sleeping state is normal is acquired by the trained model generating unit 45, and environment data indicating a room temperature of about 20 degrees suitable for sleep is acquired by the trained model generating unit 45.

In addition, a sleep log not indicating that the sleeping state is obviously abnormal but indicating that the REM sleep time or the non-REM sleep time is shorter than when the sleeping state is normal is acquired by the trained model generating unit 45, and environment data indicating a room temperature of 30 degrees or more, which is an environment where it is difficult to sleep, is acquired by the trained model generating unit 45.

Further, teacher data indicating that it is not the pre-stage state of insomnia is acquired by the trained model generating unit 45.

In a case where the sleep log, the nursing care data, the environment data, and the teacher data as described above are acquired, the trained model generating unit 45 causes the trained model 46 to perform learning so that the diagnostic data indicating that it is not the pre-stage state of insomnia is output from the trained model 46.

(6) The nursing care data in which no remarkable symptom considered to be a symptom of aspiration pneumonia is recorded is acquired by the trained model generating unit 45.

In addition, a log, which is image data indicating that a posture during a meal is a posture unsuitable for a meal and recording a coughing sound during the meal, is acquired by the trained model generating unit 45.

In addition, environment data indicating that the carbon dioxide concentration is lower than the reference concentration is acquired by the trained model generating unit 45.

Further, teacher data indicating a pre-stage state of aspiration pneumonia is acquired by the trained model generating unit 45.

In a case where the image data, the nursing care data, the environment data, and the teacher data as described above are acquired, the trained model generating unit 45 causes the trained model 46 to perform learning so that the diagnostic data indicating the pre-stage state of aspiration pneumonia is output from the trained model 46 as a presymptomatic disease in the abnormal finding absent state.

(7) The nursing care data in which no remarkable symptom considered to be a symptom of aspiration pneumonia is recorded is acquired by the trained model generating unit 45.

In addition, a log, which is image data indicating that a posture during a meal is a posture unsuitable for a meal and recording a coughing sound during the meal, is acquired by the trained model generating unit 45.

In addition, environment data indicating that the carbon dioxide concentration is higher than the reference concentration is acquired by the trained model generating unit 45.

Further, teacher data indicating that it is not the pre-stage state of aspiration pneumonia is acquired by the trained model generating unit 45.

In a case where the image data, the nursing care data, the environment data, and the teacher data as described above are acquired, the trained model generating unit 45 causes the trained model 46 to perform learning so that the diagnostic data indicating that it is not the pre-stage state of aspiration pneumonia is output from the trained model 46.

(8) The nursing care data in which no remarkable symptom considered to be a symptom of aspiration pneumonia is recorded is acquired by the trained model generating unit 45.

In addition, a log that is image data in which a coughing sound during a meal is not recorded or nursing care data in which there is no record indicating that the user may cough during a meal is acquired by the trained model generating unit 45.

In addition, environmental data indicating the carbon dioxide concentration is acquired by the trained model generating unit 45.

Further, teacher data indicating that it is not the pre-stage state of aspiration pneumonia is acquired by the trained model generating unit 45.

In a case where the image data, the nursing care data, the environment data, and the teacher data as described above are acquired, the trained model generating unit 45 causes the trained model 46 to perform learning so that the diagnostic data indicating that it is not the pre-stage state of aspiration pneumonia is output from the trained model 46.

(9) The nursing care data in which no remarkable symptom considered to be a walking disorder is recorded is acquired by the trained model generating unit 45.

In addition, a walking log not indicating that the walking state is obviously abnormal but indicating that the walking state is deteriorated is acquired by the trained model generating unit 45.

Furthermore, environment data indicating that no obstacle was present during walking is acquired by the trained model generating unit 45.

Further, teacher data indicating a pre-stage state of the walking disorder is acquired by the trained model generating unit 45.

In a case where the walking log, the nursing care data, the environment data, and the teacher data as described above are acquired, the trained model generating unit 45 causes the trained model 46 to perform learning so that the diagnostic data indicating the pre-stage state of the walking disorder is output from the trained model 46 as a presymptomatic disease in the abnormal finding absent state.

(10) No remarkable symptom considered to be a walking disorder is recorded in the nursing care data previously acquired by the trained model generating unit 45. The walking log previously acquired by the trained model generating unit 45 does not indicate that the walking state is obviously abnormal.

A remarkable symptom considered to be a walking disorder is recorded in the nursing care data recently acquired by the trained model generating unit 45. Alternatively, the walking log recently acquired by the trained model generating unit 45 indicates that the walking state is obviously abnormal.

Furthermore, environment data indicating that no obstacle was present during walking is acquired by the trained model generating unit 45.

Further, teacher data indicating a pre-stage state of the walking disorder is acquired by the trained model generating unit 45.

In a case where the walking log, the nursing care data, and the teacher data as described above are acquired, the trained model generating unit 45 causes the trained model 46 to perform learning so that diagnostic data indicating a pre-stage state of the walking disorder is output from the trained model 46 as a presymptomatic disease in the abnormal finding present state.

(11) The nursing care data in which no remarkable symptom considered to be a walking disorder is recorded is acquired by the trained model generating unit 45.

In addition, a walking log not indicating that the walking state is obviously abnormal but indicating that the walking state is deteriorated is acquired by the trained model generating unit 45.

Furthermore, environment data indicating that an obstacle was present during walking is acquired by the trained model generating unit 45.

Further, teacher data indicating that it is not the pre-stage state of the walking disorder is acquired by the trained model generating unit 45.

In a case where the walking log, the nursing care data, the environment data, and the teacher data as described above are acquired, the trained model generating unit 45 causes the trained model 46 to perform learning so that diagnostic data indicating that it is not the pre-stage state of the walking disorder is output from the trained model 46.

(12) The nursing care data in which no remarkable symptom considered to be a walking disorder is recorded is acquired by the trained model generating unit 45. In addition, nursing care data indicating that the walking amount of the person to be diagnosed exceeds the walking amount for which overwork is assumed is acquired by the trained model generating unit 45.

In addition, a walking log not indicating that the walking state is obviously abnormal but indicating that the walking state is deteriorated is acquired by the trained model generating unit 45.

In addition, environment data indicating the presence or absence of an obstacle and the like is acquired by the trained model generating unit 45.

Further, teacher data indicating that it is not the pre-stage state of the walking disorder is acquired by the trained model generating unit 45.

In a case where the walking log, the nursing care data, the environment data, and the teacher data as described above are acquired, the trained model generating unit 45 causes the trained model 46 to perform learning so that diagnostic data indicating that it is not the pre-stage state of the walking disorder is output from the trained model 46.

(13) The nursing care data in which no remarkable symptom considered to be a symptom of dementia is recorded is acquired by the trained model generating unit 45.

In addition, an operation log indicating that the same erroneous operation is repeated a predetermined number of times even when the frequency of the erroneous operation of the device is low, or nursing care data recording that the same erroneous operation is repeated a predetermined number of times even when the frequency of the erroneous operation of the device is low is acquired by the trained model generating unit 45.

Furthermore, environment data indicating that the temperature is not a dangerous temperature at which heat stroke may occur is acquired by the trained model generating unit 45.

Further, teacher data indicating that it is a pre-stage state of dementia is acquired by the trained model generating unit 45.

In a case where the operation log, the nursing care data, the environment data, and the teacher data as described above are acquired, the trained model generating unit 45 causes the trained model 46 to perform learning so that diagnostic data indicating that it is a pre-stage state of dementia is output from the trained model 46 as a presymptomatic disease in the abnormal finding present state.

(14) The nursing care data in which no remarkable symptom considered to be a symptom of dementia is recorded is acquired by the trained model generating unit 45.

In addition, an operation log indicating that the same erroneous operation is not repeated a predetermined number of times even when the frequency of the erroneous operation of the device is high, or nursing care data in which it is recorded that the same erroneous operation is not repeated a predetermined number of times even when the frequency of the erroneous operation of the device is high is acquired by the trained model generating unit 45.

Furthermore, environment data indicating the temperature of the surrounding environment is acquired by the trained model generating unit 45.

Further, teacher data indicating that it is not the pre-stage state of dementia is acquired by the trained model generating unit 45.

In a case where the sleep log, the nursing care data, the environment data, and the teacher data as described above are acquired, the trained model generating unit 45 causes the trained model 46 to perform learning so that the diagnostic data indicating that it is not the pre-stage state of dementia is output from the trained model 46.

(15) Nursing care data in which no remarkable symptom considered to be a symptom of dementia is recorded is acquired by the trained model generating unit 45.

In addition, an operation log indicating that the same erroneous operation is not repeated a predetermined number of times even when the frequency of the erroneous operation of the device is high, or nursing care data in which it is recorded that the same erroneous operation is not repeated a predetermined number of times even when the frequency of the erroneous operation of the device is high is acquired by the trained model generating unit 45.

Furthermore, environment data indicating that the temperature is a dangerous temperature at which heat stroke may occur is acquired by the trained model generating unit 45. At this time, the operation log indicating the operating operation of the air conditioner in the cooling mode is not acquired by the trained model generating unit 45. When the temperature is a dangerous temperature, it is assumed that the person to be diagnosed performs the operating operation in the cooling mode unless the person to be diagnosed is in the pre-stage state of dementia. On the other hand, when the temperature is a dangerous temperature and the person to be diagnosed does not perform the operating operation in the cooling mode, there is a high possibility that the person to be diagnosed is in a pre-stage state of dementia.

Further, teacher data indicating that it is a pre-stage state of dementia is acquired by the trained model generating unit 45.

In a case where the operation log, the nursing care data, the environment data, and the teacher data as described above are acquired, the trained model generating unit 45 causes the trained model 46 to perform learning so that diagnostic data indicating that it is a pre-stage state of dementia is output from the trained model 46 as a presymptomatic disease in the abnormal finding present state.

The trained model generating unit 45 provides the learned trained model 46 to the presymptomatic disease diagnosing unit 16 of the presymptomatic disease diagnosis device 1 illustrated in FIG. 11.

In the second embodiment described above, the data acquiring unit 44 acquires the log indicating the change in the body of the person to be diagnosed for a presymptomatic disease, acquires the nursing care data indicating the nursing care content for the person to be diagnosed for a presymptomatic disease, acquires the environment data indicating the environment around the person to be diagnosed for a presymptomatic disease, and acquires the teacher data indicating the presymptomatic disease possibly occurring in the person to be diagnosed or the teacher data indicating not the presymptomatic disease. Then, the trained model generation device 3 illustrated in FIG. 13 is configured such that the trained model generating unit 45 learns the presymptomatic disease possibly occurring in the person to be diagnosed using each of the log, the nursing care data, the environment data, and the teacher data acquired by the data acquiring unit 44, and generates the trained model 46 that outputs the diagnostic data indicating the presymptomatic disease possibly occurring in the person to be diagnosed when a log indicating the change in the body of the person to be diagnosed for a presymptomatic disease, the nursing care data indicating the nursing care content for the person to be diagnosed for a presymptomatic disease, and the environment data indicating the surrounding environment of the person to be diagnosed for a presymptomatic disease are given. Therefore, the trained model generation device 3 illustrated in FIG. 13 can provide the trained model 46 capable of improving the diagnosis accuracy of the presymptomatic disease as compared with the trained model generation device 3 illustrated in FIG. 4.

### Third Embodiment

In a third embodiment, a presymptomatic disease diagnosis device 1 including a determination unit 18 that determines whether an environment around a person to be diagnosed is a normal environment or an abnormal environment will be described.

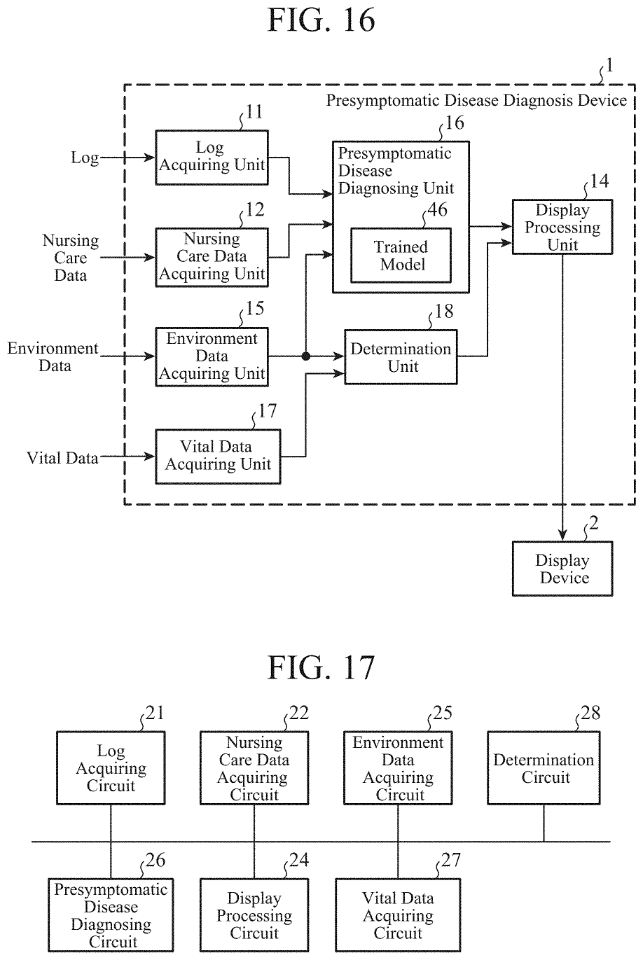

FIG. 16 is a configuration diagram illustrating a presymptomatic disease diagnosis device 1 according to the third embodiment.

FIG. 17 is a hardware configuration diagram illustrating hardware of the presymptomatic disease diagnosis device 1 according to the third embodiment. In FIGS. 16 and 17, the same reference numerals as those in FIGS. 1, 2, 11, and 12 denote the same or corresponding parts, and thus description thereof is omitted.

The presymptomatic disease diagnosis device 1 illustrated in FIG. 16 includes a log acquiring unit 11, a nursing care data acquiring unit 12, an environment data acquiring unit 15, a presymptomatic disease diagnosing unit 16, a display processing unit 14, a vital data acquiring unit 17, and a determination unit 18.

In the presymptomatic disease diagnosis device 1 illustrated in FIG. 16, the vital data acquiring unit 17 and the determination unit 18 are applied to the presymptomatic disease diagnosis device 1 illustrated in FIG. 11. However, this is merely an example, and the vital data acquiring unit 17 and the determination unit 18 may be applied to the presymptomatic disease diagnosis device 1 illustrated in FIG. 1.

The vital data acquiring unit 17 is implemented by, for example, a vital data acquiring circuit 27 shown in FIG. 17.

The vital data acquiring unit 17 acquires vital data indicating vitals of the person to be diagnosed or vital data indicating vitals of a staff who cares for the person to be diagnosed.

The vital data acquiring unit 17 outputs the vital data to the determination unit 18.

The determination unit 18 is implemented by, for example, a determination circuit 28 illustrated in FIG. 17.

The determination unit 18 compares the boundary data indicating the boundary between the normal environment and the abnormal environment around the person to be diagnosed with the environment data acquired by the environment data acquiring unit 15. When the environment data acquired by the environment data acquiring unit 15 is, for example, data indicating a temperature, the determination unit 18 acquires boundary data indicating a boundary between a normal ambient temperature and an abnormal temperature around the person to be diagnosed. As the boundary data, for example, data indicating a temperature of about 32 degrees is conceivable for the purpose of heat stroke prevention. As the boundary data, for example, data indicating a temperature of about 8 degrees is conceivable for the purpose of hypothermia prevention. As the boundary data, for example, data indicating a carbon dioxide concentration of about 3% is conceivable for the purpose of carbon dioxide poisoning prevention. The boundary data may be stored in the internal memory of the determination unit 18, or may be provided from the outside of the presymptomatic disease diagnosis device 1.

The determination unit 18 determines whether the environment around the person to be diagnosed is a normal environment or an abnormal environment on the basis of the comparison result between the boundary data and the environment data.

In addition, the determination unit 18 compares the vital data indicating the vitals of the person to be diagnosed acquired by the vital data acquiring unit 17 with a threshold value Th₁, and determines whether the vitals of the person to be diagnosed are normal or abnormal on the basis of the comparison result between the vital data and the threshold value Th₁.

In addition, the determination unit 18 compares the vital data indicating the vitals of the staff acquired by the vital data acquiring unit 17 with a threshold value Th₂, and determines whether the vitals of the staff are normal or abnormal on the basis of the comparison result between the vital data and the threshold value Th₂.

The determination unit 18 outputs a determination result indicating whether it is normal or abnormal to the display processing unit 14.

The threshold values Th₁ and Th₂ may be stored in the internal memory of the determination unit 18, or may be given from the outside of the presymptomatic disease diagnosis device 1. The threshold value Th₁ and the threshold value Th₂ may be the same value or different values from each other.

In FIG. 16, it is assumed that each of the log acquiring unit 11, the nursing care data acquiring unit 12, the environment data acquiring unit 15, the presymptomatic disease diagnosing unit 16, the display processing unit 14, the vital data acquiring unit 17, and the determination unit 18, which are components of the presymptomatic disease diagnosis device 1, is implemented by dedicated hardware as illustrated in FIG. 17. That is, it is assumed that the presymptomatic disease diagnosis device 1 is implemented by the log acquiring circuit 21, the nursing care data acquiring circuit 22, the environment data acquiring circuit 25, the presymptomatic disease diagnosing circuit 26, the display processing circuit 24, the vital data acquiring circuit 27, and the determination circuit 28.

Each of the log acquiring circuit 21, the nursing care data acquiring circuit 22, the environment data acquiring circuit 25, the presymptomatic disease diagnosing circuit 26, the display processing circuit 24, the vital data acquiring circuit 27, and the determination circuit 28 corresponds to, for example, a single circuit, a composite circuit, a programmed processor, a parallel-programmed processor, ASIC, FPGA, or a combination thereof.

The components of the presymptomatic disease diagnosis device 1 are not limited to those implemented by dedicated hardware, and the presymptomatic disease diagnosis device 1 may be implemented by software, firmware, or a combination of software and firmware.

In a case where the presymptomatic disease diagnosis device 1 is implemented by software, firmware, or the like, a program for causing a computer to execute each processing procedure in the log acquiring unit 11, the nursing care data acquiring unit 12, the environment data acquiring unit 15, the presymptomatic disease diagnosing unit 16, the display processing unit 14, the vital data acquiring unit 17, and the determination unit 18 is stored in the memory 31 illustrated in FIG. 3. Then, the processor 32 illustrated in FIG. 3 executes the program stored in the memory 31.

In addition, FIG. 17 illustrates an example in which each of the components of the presymptomatic disease diagnosis device 1 is implemented by dedicated hardware, and FIG. 3 illustrates an example in which the presymptomatic disease diagnosis device 1 is implemented by software, firmware, or the like. However, this is merely an example, and some of the components in the presymptomatic disease diagnosis device 1 may be implemented by dedicated hardware, and the remaining components may be implemented by software, firmware, or the like.

Next, the operation of the presymptomatic disease diagnosis device 1 illustrated in FIG. 16 will be described.

Since the units other than the vital data acquiring unit 17 and the determination unit 18 are similar to those of the presymptomatic disease diagnosis device 1 shown in FIG. 11, the operations of the vital data acquiring unit 17 and the determination unit 18 will be mainly described here.

The vital data acquiring unit 17 acquires vital data indicating the vitals of the person to be diagnosed from a vital sensor attached to the person to be diagnosed.

In addition, the vital data acquiring unit 17 acquires vital data indicating the vitals of a staff who cares for the person to be diagnosed from a vital sensor attached to the staff.

The vital data acquiring unit 17 outputs the vital data to the determination unit 18.

In the presymptomatic disease diagnosis device 1 shown in FIG. 16, the vital data acquiring unit 17 acquires vital data from the vital sensor. However, this is merely an example, and the vital data acquiring unit 17 may acquire the vital data from a computer or the like that manages the vitals of the person to be diagnosed or the vitals of the staff.

The determination unit 18 acquires the environment data from the environment data acquiring unit 15.

The determination unit 18 compares the boundary data stored in the internal memory or the like with the environment data acquired by the environment data acquiring unit 15.

The determination unit 18 determines whether the environment around the person to be diagnosed is a normal environment or an abnormal environment on the basis of the comparison result between the boundary data and the environment data.

For example, for the purpose of heat stroke prevention, when boundary data indicating a temperature of about 32 degrees is stored in an internal memory or the like, if the temperature indicated by the environment data is equal to or higher than the boundary data, the determination unit 18 determines that the environment around the person to be diagnosed is an abnormal environment. If the temperature indicated by the environment data is less than the boundary data, the determination unit 18 determines that the environment around the person to be diagnosed is a normal environment.

For example, for the purpose of hypothermia prevention, when boundary data indicating a temperature of about 8 degrees is stored in an internal memory or the like, if the temperature indicated by the environment data is equal to or lower than the boundary data, the determination unit 18 determines that the environment around the person to be diagnosed is an abnormal environment. If the temperature indicated by the environment data is more than the boundary data, the determination unit 18 determines that the environment around the person to be diagnosed is a normal environment.

For example, for the purpose of carbon dioxide poisoning prevention, when boundary data indicating a carbon dioxide concentration of about 3% is stored in an internal memory or the like, if the carbon dioxide concentration indicated by the environment data is equal to or more than the boundary data, the determination unit 18 determines that the environment around the person to be diagnosed is an abnormal environment. If the carbon dioxide concentration indicated by the environment data is less than the boundary data, the determination unit 18 determines that the environment around the person to be diagnosed is a normal environment.

The determination unit 18 outputs a determination result indicating whether the environment around the person to be diagnosed is normal or abnormal to the display processing unit 14.

The display processing unit 14 acquires, from the determination unit 18, the determination result indicating whether the environment around the person to be diagnosed is normal or abnormal.

The display processing unit 14 generates display data for displaying a place where an abnormality occurs on the basis of the acquired determination result. The place where the abnormality occurs is the installation position of the sensor indicated by the position data included in the environment data.

The display processing unit 14 outputs the display data to the display device 2. The display device 2 displays the place where the abnormality occurs on the screen according to the display data output from the display processing unit 14.

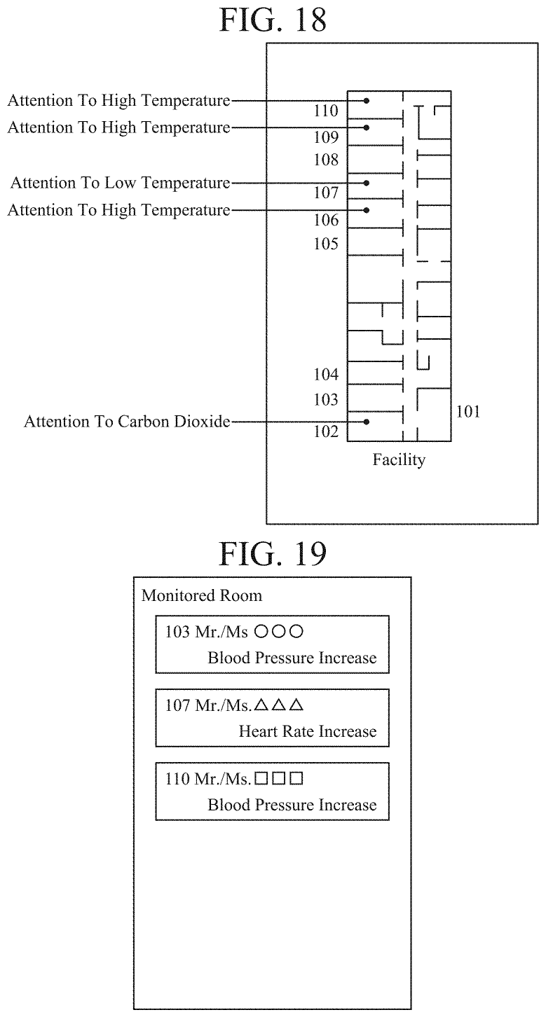

FIG. 18 is an explanatory diagram illustrating an example of a place where an abnormality occurs in a facility.

In FIG. 18, a place denoted by “ATTENTION TO HIGH TEMPERATURE” indicates the position of the abnormal environment where the temperature indicated by the environment data is higher than the temperature indicated by the boundary data.

A place with “ATTENTION TO LOW TEMPERATURE” indicates a position of the abnormal environment where the temperature indicated by the environment data is lower than the temperature indicated by the boundary data.

A place with “ATTENTION TO CARBON DIOXIDE” indicates a position of the abnormal environment where the carbon dioxide concentration indicated by the environment data is higher than the carbon dioxide concentration indicated by the boundary data.

In the example of FIG. 18, the temperatures in the room 106, the room 109, and the room 110 in the facility are high. The temperature in the room 107 in the facility is low. The carbon dioxide concentration in the room 102 in the facility is high.

The determination unit 18 acquires, from the vital data acquiring unit 17, vital data indicating the vitals of the person to be diagnosed.

The determination unit 18 compares the vital data indicating the vitals of the person to be diagnosed with the threshold value Th₁.

The determination unit 18 determines whether the vitals of the person to be diagnosed are normal or abnormal on the basis of the comparison result between the vital data and the threshold value Th₁.

For example, when the vital data is data indicating blood pressure and the blood pressure indicating the boundary between a normal blood pressure and a high blood pressure is stored as the threshold value Th₁ in an internal memory or the like, if the vital data is equal to or more than the threshold value Th₁, the determination unit 18 determines that the vitals of the person to be diagnosed are abnormal. If the vital data is less than the threshold value Th₁, the determination unit 18 determines that the vitals of the person to be diagnosed are normal.

For example, when the vital data is data indicating a heart rate and an upper limit value of a normal heart rate is stored as the threshold value Th₁ in an internal memory or the like, if the vital data is equal to or more than the threshold value Th₁, the determination unit 18 determines that the vitals of the person to be diagnosed are abnormal. If the vital data is less than the threshold value Th₁, the determination unit 18 determines that the vitals of the person to be diagnosed are normal.

The determination unit 18 outputs a determination result indicating whether the vitals of the person to be diagnosed are normal or abnormal to the display processing unit 14.

The display processing unit 14 acquires a determination result indicating whether the vitals of the person to be diagnosed are normal or abnormal from the determination unit 18.

The display processing unit 14 generates display data for displaying a person to be diagnosed whose vitals are abnormal on the basis of the acquired determination result.

The display processing unit 14 outputs the display data to the display device 2. The display device 2 displays the person to be diagnosed whose vitals are abnormal on the screen according to the display data output from the display processing unit 14.

FIG. 19 is an explanatory diagram illustrating a list of persons to be diagnosed whose vitals are abnormal.

The example of FIG. 19 shows that there is a vital abnormality in a person to be diagnosed who lives in each of the room 103, the room 107, and the room 110 in the facility.

In FIG. 19, “BLOOD PRESSURE INCREASE” indicates that the blood pressure of the person to be diagnosed is equal to or more than the upper limit value of the normal blood pressure, and “HEART RATE INCREASE” indicates that the heart rate of the person to be diagnosed is equal to or more than the upper limit value of the normal heart rate.

The determination unit 18 acquires, from the vital data acquiring unit 17, vital data indicating the vitals of the staff.

The determination unit 18 compares the vital data indicating the vitals of the staff with the threshold value Th₂.

The determination unit 18 determines whether the vitals of the staff is normal or abnormal on the basis of the comparison result between the vital data and the threshold value Th₂.

The determination unit 18 outputs a determination result indicating whether the vitals of the staff are normal or abnormal to the display processing unit 14.

The display processing unit 14 acquires a determination result indicating whether the vitals of the staff are normal or abnormal from the determination unit 18.

The display processing unit 14 generates display data for displaying a staff whose vitals are abnormal on the basis of the acquired determination result.

The display processing unit 14 outputs the display data to the display device 2.

The display device 2 displays the staff whose vitals are abnormal on the screen according to the display data output from the display processing unit 14. For example, the name of the staff whose vitals are abnormal and the item of the abnormal vital are displayed on the screen.

Here, an example is illustrated in which the display device 2 displays a name of a staff whose vitals are abnormal and an item of the abnormal vital on a screen. However, this is merely an example, and for example, if the staff carries a global positioning system (GPS) sensor, the display processing unit 14 may specify the position of the staff on the basis of the position information output from the GPS sensor, and generate display data for displaying the position of the staff on the map. As a result, it is possible to check where the staff in which the abnormality occurs in the vitals is.

In addition, the display processing unit 14 may generate display data for displaying the nursing care content indicated by the nursing care data acquired by the nursing care data acquiring unit 12 together with the position of the staff. As a result, the staff can check where and what kind of nursing care the staff is providing.

Furthermore, the display processing unit 14 may generate display data for displaying a history of nursing care contents by a staff in a list. As a result, it is possible to easily check the nursing care content by the staff.

In the third embodiment described above, the presymptomatic disease diagnosis device 1 illustrated in FIG. 16 is configured to include the determination unit 18 to compare boundary data indicating a boundary between a normal environment and an abnormal environment around the person to be diagnosed with the environment data acquired by the environment data acquiring unit 15, and determines whether the environment around the person to be diagnosed is a normal environment or an abnormal environment on the basis of the comparison result between the boundary data and the environment data. Therefore, as with the presymptomatic disease diagnosis device 1 illustrated in FIG. 1, the presymptomatic disease diagnosis device 1 illustrated in FIG. 16 can diagnose a presymptomatic disease in the abnormal finding absent state, and can check whether the environment around the person to be diagnosed is normal or abnormal.

In the third embodiment described above, the presymptomatic disease diagnosis device 1 illustrated in FIG. 16 is configured to include the vital data acquiring unit 17 to acquire vital data indicating the vitals of the person to be diagnosed, and the determination unit 18 to compare the vital data acquired by the vital data acquiring unit 17 with a threshold value, and determine whether the vitals of the person to be diagnosed are normal or abnormal on the basis of the comparison result between the vital data and the threshold value. Therefore, the presymptomatic disease diagnosis device 1 illustrated in FIG. 16 can check whether the vitals of the person to be diagnosed are normal or abnormal.

In the third embodiment described above, the presymptomatic disease diagnosis device 1 illustrated in FIG. 16 is configured to include the vital data acquiring unit 17 to acquire vital data indicating vitals of a staff who cares for the person to be diagnosed; and the determination unit 18 to compare the vital data acquired by the vital data acquiring unit 17 with a threshold value, and determine whether the vitals of the staff are normal or abnormal on the basis of the comparison result between the vital data and the threshold value. Therefore, the presymptomatic disease diagnosis device 1 illustrated in FIG. 16 can check whether the vitals of the staff are normal or abnormal.

### Fourth Embodiment

In a fourth embodiment, a presymptomatic disease diagnosis device 1 including a display data generating unit 19 that generates display data for displaying a position or the like where a plurality of sensors for observing an environment around a person to be diagnosed are installed will be described.

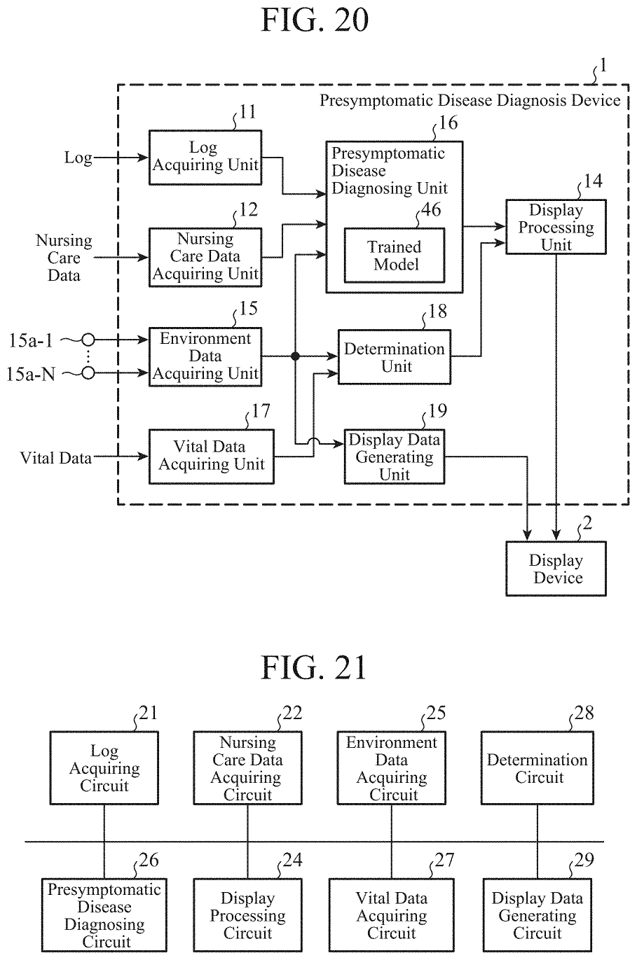

FIG. 20 is a configuration diagram illustrating a presymptomatic disease diagnosis device 1 according to a fourth embodiment.

FIG. 21 is a hardware configuration diagram illustrating hardware of the presymptomatic disease diagnosis device 1 according to the fourth embodiment. In FIGS. 20 and 21, the same reference numerals as those in FIGS. 1, 2, 11, 12, 16, and 17 denote the same or corresponding parts, and thus description thereof is omitted.

The presymptomatic disease diagnosis device 1 illustrated in FIG. 20 includes a log acquiring unit 11, a nursing care data acquiring unit 12, an environment data acquiring unit 15, a presymptomatic disease diagnosing unit 16, a display processing unit 14, a vital data acquiring unit 17, a determination unit 18, and a display data generating unit 19.

In the presymptomatic disease diagnosis device 1 illustrated in FIG. 20, the display data generating unit 19 is applied to the presymptomatic disease diagnosis device 1 illustrated in FIG. 16. However, this is merely an example, and the display data generating unit 19 may be applied to the presymptomatic disease diagnosis device 1 illustrated in FIG. 1 or the presymptomatic disease diagnosis device 1 illustrated in FIG. 11.

The environment data acquiring unit 15 acquires environment data indicating an observation result of the environment from each of the plurality of sensors 15a-1, . . . , 15a-N that observes the environment around the person to be diagnosed. N is an integer of 2 or more.

Examples of the sensor 15a-n (n=1, . . . , N) include a room temperature sensor, a humidity sensor, an illuminance sensor, an atmospheric pressure sensor, a carbon dioxide sensor, a pollution observation sensor, an odor sensor, and a monitoring camera.

The display data generating unit 19 is implemented by, for example, a display data generating circuit 29 illustrated in FIG. 21.

The display data generating unit 19 generates display data for displaying the position where the sensor 15a-n is installed and the environment data output from the sensor 15a-n on the screen.

The display data generating unit 19 outputs the display data to the display device 2.

In FIG. 20, it is assumed that each of the log acquiring unit 11, the nursing care data acquiring unit 12, the environment data acquiring unit 15, the presymptomatic disease diagnosing unit 16, the display processing unit 14, the vital data acquiring unit 17, the determination unit 18, and the display data generating unit 19, which are components of the presymptomatic disease diagnosis device 1, is implemented by dedicated hardware as illustrated in FIG. 21. That is, it is assumed that the presymptomatic disease diagnosis device 1 is implemented by the log acquiring circuit 21, the nursing care data acquiring circuit 22, the environment data acquiring circuit 25, the presymptomatic disease diagnosing circuit 26, the display processing circuit 24, the vital data acquiring circuit 27, the determination circuit 28, and the display data generating circuit 29.

Each of the log acquiring circuit 21, the nursing care data acquiring circuit 22, the environment data acquiring circuit 25, the presymptomatic disease diagnosing circuit 26, the display processing circuit 24, the vital data acquiring circuit 27, the determination circuit 28, and the display data generating circuit 29 corresponds to, for example, a single circuit, a composite circuit, a programmed processor, a parallel-programmed processor, ASIC, FPGA, or a combination thereof.

The components of the presymptomatic disease diagnosis device 1 are not limited to those implemented by dedicated hardware, and the presymptomatic disease diagnosis device 1 may be implemented by software, firmware, or a combination of software and firmware.

In a case where the presymptomatic disease diagnosis device 1 is implemented by software, firmware, or the like, a program for causing a computer to execute each processing procedure in the log acquiring unit 11, the nursing care data acquiring unit 12, the environment data acquiring unit 15, the presymptomatic disease diagnosing unit 16, the display processing unit 14, the vital data acquiring unit 17, the determination unit 18, and the display data generating unit 19 is stored in the memory 31 illustrated in FIG. 3. Then, the processor 32 illustrated in FIG. 3 executes the program stored in the memory 31.

Next, the operation of the presymptomatic disease diagnosis device 1 illustrated in FIG. 20 will be described.

Since the units other than the display data generating unit 19 are similar to those of the presymptomatic disease diagnosis device 1 illustrated in FIG. 16, the operation of the display data generating unit 19 will be mainly described here.

The display data generating unit 19 acquires the environment data output from the sensor 15a-n included in the environment data acquiring unit 15. The environment data includes position data indicating the installation position of the sensor 15a-n.

In the presymptomatic disease diagnosis device 1 illustrated in FIG. 20, the environment data includes position data indicating the installation position of the sensor 15a-n. However, this is merely an example, and the internal memory of the display data generating unit 19 may store position data indicating the installation position of the sensor 15a-n.

The display data generating unit 19 generates display data for displaying the position where the sensor 15a-n is installed and the environment data output from the sensor 15a-n on the screen.

The display data generating unit 19 outputs the display data to the display device 2.

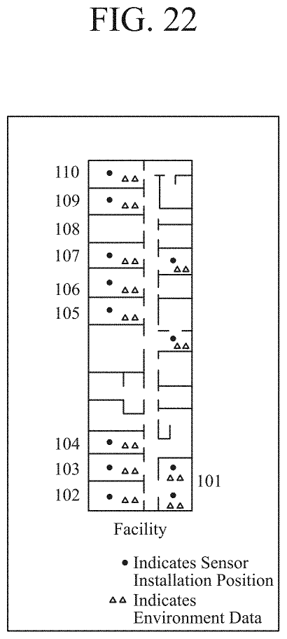

As illustrated in FIG. 22, the display device 2 displays the position where the sensor 15a-n is installed and the environment data output from the sensor 15a-n on the screen according to the display data output from the display data generating unit 19.

FIG. 22 is an explanatory diagram illustrating a display example of the position where the sensor 15a-n is installed and the environment data output from the sensor 15 a-n.

In FIG. 22, “●” indicates the position where the sensor 15a-n is installed, and “ΔΔ” indicates the environment data output from the sensor 15a-n.

In the above-described fourth embodiment, the presymptomatic disease diagnosis device 1 illustrated in FIG. 20 is configured such that the environment data acquiring unit 15 acquires environment data indicating an observation result of an environment from each of a plurality of sensors 15a-1 to 15a-N that observes an environment around the person to be diagnosed, and includes a display data generating unit 19 to generate display data for displaying a position where each sensor 15a-n is installed and environment data output from each sensor 15a-n on the screen. Therefore, similarly to the presymptomatic disease diagnosis device illustrated in FIG. 1, the presymptomatic disease diagnosis device illustrated in FIG. 20 can diagnose a presymptomatic disease in the abnormal finding absent state, and can check the position where the sensor 15a-n is installed and the environment data output from the sensor 15 a-n.

### Fifth Embodiment

In a fifth embodiment, a presymptomatic disease diagnosis device 1 including a display data generating unit 72 that generates display data for displaying movement of a skeleton of a person to be diagnosed on the screen according to skeleton data output from a skeleton analysis unit 71 will be described.

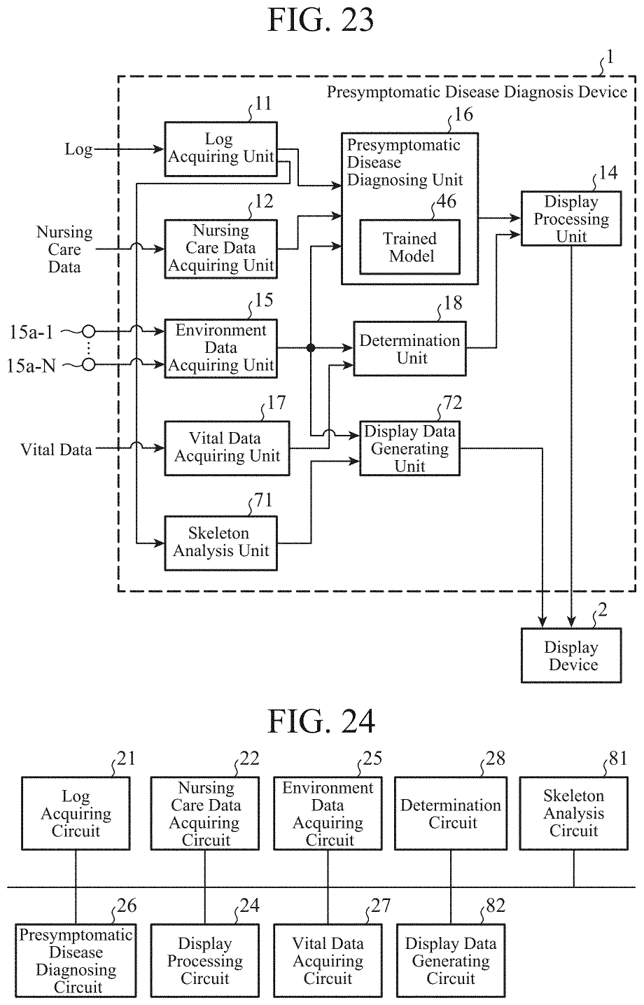

FIG. 23 is a configuration diagram illustrating a presymptomatic disease diagnosis device 1 according to the fifth embodiment.

FIG. 24 is a hardware configuration diagram illustrating hardware of the presymptomatic disease diagnosis device 1 according to the fifth embodiment. In FIGS. 23 and 24, the same reference numerals as those in FIGS. 1, 2, 11, 12, 16, 17, 20, and 21 denote the same or corresponding parts, and thus description thereof is omitted.

The presymptomatic disease diagnosis device 1 illustrated in FIG. 23 includes a log acquiring unit 11, a nursing care data acquiring unit 12, an environment data acquiring unit 15, a presymptomatic disease diagnosing unit 16, a display processing unit 14, a vital data acquiring unit 17, a determination unit 18, a skeleton analysis unit 71, and a display data generating unit 72.

In the presymptomatic disease diagnosis device 1 illustrated in FIG. 23, the skeleton analysis unit 71 and the display data generating unit 72 are applied to the presymptomatic disease diagnosis device 1 illustrated in FIG. 20. However, this is merely an example, and the skeleton analysis unit 71 and the display data generating unit 72 may be applied to the presymptomatic disease diagnosis device 1 illustrated in FIG. 1, the presymptomatic disease diagnosis device 1 illustrated in FIG. 11, or the presymptomatic disease diagnosis device 1 illustrated in FIG. 16.

In the presymptomatic disease diagnosis device 1 illustrated in FIG. 23, the log acquiring unit 11 acquires video data of a camera photographing a person to be diagnosed as a log indicating a change in the body of the person to be diagnosed.

The skeleton analysis unit 71 is implemented by, for example, a skeleton analysis circuit 81 illustrated in FIG. 24.

The skeleton analysis unit 71 analyzes the movement of the skeleton of the person to be diagnosed from the video data acquired by the log acquiring unit 11. The processing itself of analyzing the movement of the skeleton to generate skeleton data indicating the movement of the skeleton is a known technique, and thus a detailed description thereof will be omitted.

The skeleton analysis unit 71 outputs the skeleton data indicating the movement of the skeleton of the person to be diagnosed to the display data generating unit 72.

The display data generating unit 72 is implemented by, for example, a display data generating circuit 82 illustrated in FIG. 24.

Similarly to the display data generating unit 19 illustrated in FIG. 20, the display data generating unit 72 generates display data for displaying the position where the sensor 15a-n is installed and the environment data output from the sensor 15a-n on the screen.

In addition, the display data generating unit 72 generates display data for displaying the movement of the skeleton of the person to be diagnosed on the screen according to the skeleton data output from the skeleton analysis unit 71.

The display data generating unit 72 outputs the display data to the display device 2.

In FIG. 23, it is assumed that each of the log acquiring unit 11, the nursing care data acquiring unit 12, the environment data acquiring unit 15, the presymptomatic disease diagnosing unit 16, the display processing unit 14, the vital data acquiring unit 17, the determination unit 18, the skeleton analysis unit 71, and the display data generating unit 72, which are components of the presymptomatic disease diagnosis device 1, is implemented by dedicated hardware as illustrated in FIG. 24. That is, it is assumed that the presymptomatic disease diagnosis device 1 is implemented by the log acquiring circuit 21, the nursing care data acquiring circuit 22, the environment data acquiring circuit 25, the presymptomatic disease diagnosing circuit 26, the display processing circuit 24, the vital data acquiring circuit 27, the determination circuit 28, the skeleton analysis circuit 81, and the display data generating circuit 82.

Each of the log acquiring circuit 21, the nursing care data acquiring circuit 22, the environment data acquiring circuit 25, the presymptomatic disease diagnosing circuit 26, the display processing circuit 24, the vital data acquiring circuit 27, the determination circuit 28, the skeleton analysis circuit 81, and the display data generating circuit 82 corresponds to, for example, a single circuit, a composite circuit, a programmed processor, a parallel-programmed processor, ASIC, FPGA, or a combination thereof.

The components of the presymptomatic disease diagnosis device 1 are not limited to those implemented by dedicated hardware, and the presymptomatic disease diagnosis device 1 may be implemented by software, firmware, or a combination of software and firmware.

In a case where the presymptomatic disease diagnosis device 1 is implemented by software, firmware, or the like, a program for causing a computer to execute each processing procedure in the log acquiring unit 11, the nursing care data acquiring unit 12, the environment data acquiring unit 15, the presymptomatic disease diagnosing unit 16, the display processing unit 14, the vital data acquiring unit 17, the determination unit 18, the skeleton analysis unit 71, and the display data generating unit 72 is stored in the memory 31 illustrated in FIG. 3. Then, the processor 32 illustrated in FIG. 3 executes the program stored in the memory 31.

Next, the operation of the presymptomatic disease diagnosis device 1 illustrated in FIG. 23 will be described.

Since the units other than the skeleton analysis unit 71 and the display data generating unit 72 are similar to those of the presymptomatic disease diagnosis device 1 illustrated in FIG. 20, the operations of the skeleton analysis unit 71 and the display data generating unit 72 will be mainly described here.

The log acquiring unit 11 acquires video data of a camera photographing the person to be diagnosed, and outputs the video data of the camera to the skeleton analysis unit 71.

The skeleton analysis unit 71 acquires the video data of the camera from the log acquiring unit 11.

The skeleton analysis unit 71 analyzes the movement of the skeleton of the person to be diagnosed from the video data of the camera and generates skeleton data indicating the movement of the skeleton.

The skeleton analysis unit 71 outputs the skeleton data indicating the movement of the skeleton of the person to be diagnosed to the display data generating unit 72.

The display data generating unit 72 acquires skeleton data from the skeleton analysis unit 71.

The display data generating unit 72 generates display data for displaying the movement of the skeleton of the person to be diagnosed on the screen according to the skeleton data.

The display data generating unit 19 outputs the display data to the display device 2.

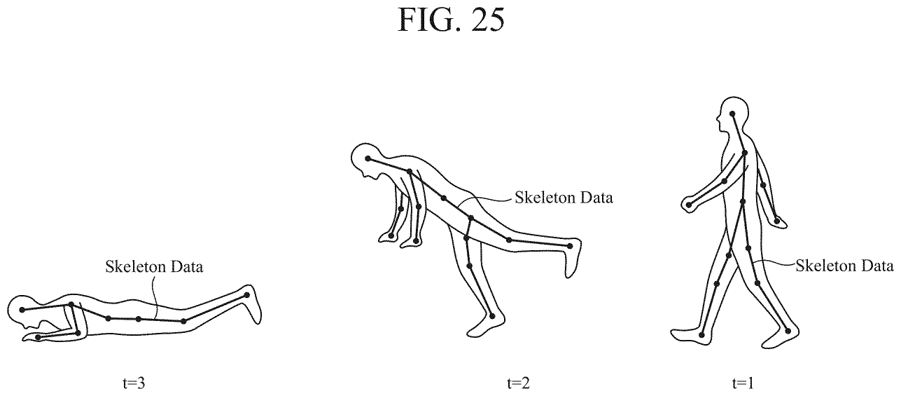

As illustrated in FIG. 25, the display device 2 displays the movement of the skeleton of the person to be diagnosed on the screen according to the display data output from the display data generating unit 19.

FIG. 25 is an explanatory diagram illustrating movement of the skeleton of the person to be diagnosed.

The example of FIG. 25 illustrates the skeleton of the person to be diagnosed at time t=1, t=2, and t=3. At t=1, the person to be diagnosed is walking normally, but at t=2, the person to be diagnosed is about to roll, and at t=3, the person to be diagnosed falls.

In the above-described embodiment 5, the presymptomatic disease diagnosis device 1 illustrated in FIG. 23 is configured such that the log acquiring unit 11 acquires, as the log indicating a change in the body of the person to be diagnosed, video data of a camera photographing the person to be diagnosed, and includes: the skeleton analysis unit 71 to analyze movement of a skeleton of the person to be diagnosed from the video data acquired by the log acquiring unit 11, and output skeleton data indicating the movement of the skeleton of the person to be diagnosed; and the display data generating unit 72 to generate display data for displaying the movement of the skeleton of the person to be diagnosed on the screen according to the skeleton data output from the skeleton analysis unit 71. Therefore, similarly to the presymptomatic disease diagnosis device illustrated in FIG. 1, the presymptomatic disease diagnosis device illustrated in FIG. 23 can diagnose the presymptomatic disease in the abnormal finding absent state and can check the movement of the skeleton of the person to be diagnosed.

### Sixth Embodiment

In a sixth embodiment, a presymptomatic disease diagnosis device 1 including a display data generating unit 73 that generates display data for displaying a change in a sleeping state indicated by a log acquired by a log acquiring unit 11 and an operation status of an air conditioner indicated by environment data acquired by an environment data acquiring unit 15 on a screen will be described.

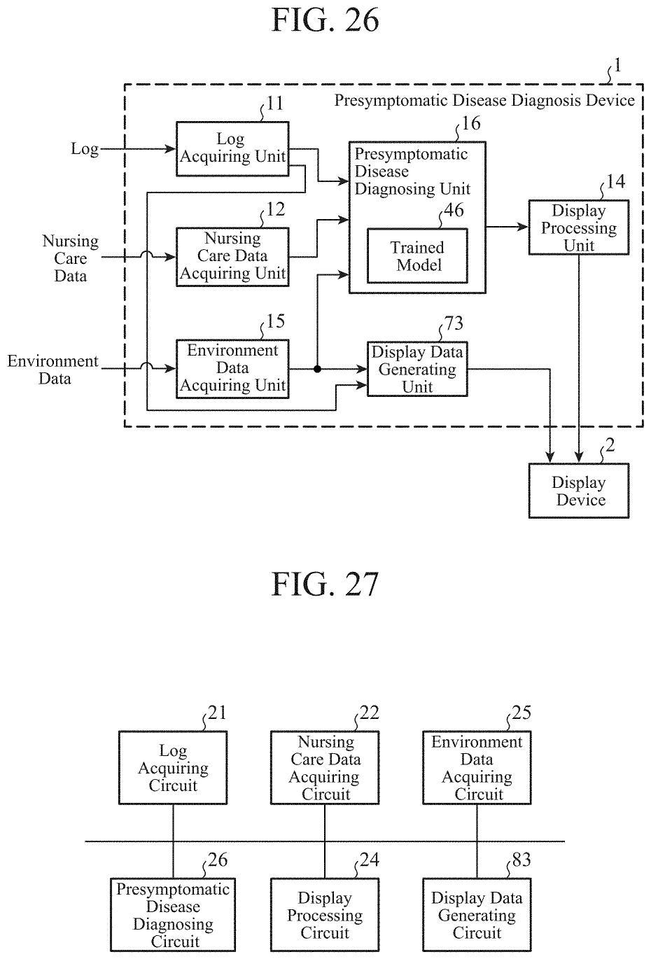

FIG. 26 is a configuration diagram illustrating a presymptomatic disease diagnosis device 1 according to a sixth embodiment.

FIG. 27 is a hardware configuration diagram illustrating hardware of the presymptomatic disease diagnosis device 1 according to the sixth embodiment. In FIGS. 26 and 27, the same reference numerals as those in FIGS. 1, 2, 11, 12, 16, 17, 20, 21, 23, and 24 denote the same or corresponding parts, and thus description thereof is omitted.

The presymptomatic disease diagnosis device 1 illustrated in FIG. 26 includes a log acquiring unit 11, a nursing care data acquiring unit 12, an environment data acquiring unit 15, a presymptomatic disease diagnosing unit 16, a display processing unit 14, and a display data generating unit 73.

In the presymptomatic disease diagnosis device 1 illustrated in FIG. 26, the display data generating unit 73 is applied to the presymptomatic disease diagnosis device 1 illustrated in FIG. 11. However, this is merely an example, and the display data generating unit 73 may be applied to the presymptomatic disease diagnosis device 1 illustrated in FIG. 1, the presymptomatic disease diagnosis device 1 illustrated in FIG. 16, the presymptomatic disease diagnosis device 1 illustrated in FIG. 20, or the presymptomatic disease diagnosis device 1 illustrated in FIG. 23.

In the presymptomatic disease diagnosis device 1 illustrated in FIG. 26, the log acquiring unit 11 acquires a sleep log indicating a change in the sleeping state of the person to be diagnosed as a log indicating a change in the body of the person to be diagnosed.

In the presymptomatic disease diagnosis device 1 illustrated in FIG. 26, the environment data acquiring unit 15 acquires environment data indicating the operation status of the air conditioner as environment data indicating the environment around the person to be diagnosed.

The display data generating unit 73 is implemented by, for example, a display data generating circuit 83 illustrated in FIG. 27.

The display data generating unit 73 generates display data for displaying the change in the sleeping state indicated by the sleep log acquired by the log acquiring unit 11 and the operation status of the air conditioner indicated by the environment data acquired by the environment data acquiring unit 15 on the screen.

The display data generating unit 73 outputs the display data to the display device 2.

In FIG. 26, it is assumed that each of the log acquiring unit 11, the nursing care data acquiring unit 12, the environment data acquiring unit 15, the presymptomatic disease diagnosing unit 16, the display processing unit 14, and the display data generating unit 73, which are components of the presymptomatic disease diagnosis device 1, is implemented by dedicated hardware as illustrated in FIG. 27. That is, it is assumed that the presymptomatic disease diagnosis device 1 is implemented by the log acquiring circuit 21, the nursing care data acquiring circuit 22, the environment data acquiring circuit 25, the presymptomatic disease diagnosing circuit 26, the display processing circuit 24, and the display data generating circuit 83.

Each of the log acquiring circuit 21, the nursing care data acquiring circuit 22, the environment data acquiring circuit 25, the presymptomatic disease diagnosing circuit 26, the display processing circuit 24, and the display data generating circuit 83 corresponds to, for example, a single circuit, a composite circuit, a programmed processor, a parallel-programmed processor, ASIC, FPGA, or a combination thereof.

The components of the presymptomatic disease diagnosis device 1 are not limited to those implemented by dedicated hardware, and the presymptomatic disease diagnosis device 1 may be implemented by software, firmware, or a combination of software and firmware.

In a case where the presymptomatic disease diagnosis device 1 is implemented by software, firmware, or the like, a program for causing a computer to execute each processing procedure in the log acquiring unit 11, the nursing care data acquiring unit 12, the environment data acquiring unit 15, the presymptomatic disease diagnosing unit 16, the display processing unit 14, and the display data generating unit 73 is stored in the memory 31 illustrated in FIG. 3. Then, the processor 32 illustrated in FIG. 3 executes the program stored in the memory 31.

Next, the operation of the presymptomatic disease diagnosis device 1 illustrated in FIG. 26 will be described.

Since the units other than the display data generating unit 73 are similar to the presymptomatic disease diagnosis device 1 illustrated in FIG. 11, the operation of the display data generating unit 73 will be mainly described here.

In the presymptomatic disease diagnosis device 1 illustrated in FIG. 26, the log acquiring unit 11 acquires a sleep log indicating a change in the sleeping state of the person to be diagnosed as a log indicating a change in the body of the person to be diagnosed.

The log acquiring unit 11 outputs the sleep log to the display data generating unit 73.

The environment data acquiring unit 15 acquires environment data indicating the operation status of the air conditioner as environment data indicating the environment around the person to be diagnosed.

The environment data acquiring unit 15 outputs the environment data indicating the operation status of the air conditioner to the display data generating unit 73.

The display data generating unit 73 acquires the sleep log from the log acquiring unit 11, and acquires the environment data indicating the operation status of the air conditioner from the environment data acquiring unit 15.

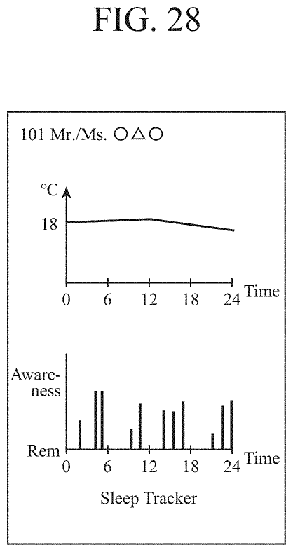

As illustrated in FIG. 28, the display data generating unit 73 generates display data for displaying a sleep tracker indicating a change in the sleeping state indicated by the sleep log and the operation status of the air conditioner indicated by the environment data on the screen.

The display data generating unit 73 outputs the display data to the display device 2.

The display device 2 displays a change in the sleeping state and the operation status of the air conditioner on the screen according to the display data output from the display data generating unit 73.

FIG. 28 is an explanatory diagram illustrating a change in the sleeping state and an operation status of the air conditioner.

The example of FIG. 28 illustrates a sleep tracker indicating a change in the sleeping state of “Mr. ◯Δ◯” in the room No. 101 among the plurality of persons to be diagnosed and an operation status of an air conditioner in the room No. 101.

In the above-described sixth embodiment, the presymptomatic disease diagnosis device 1 illustrated in FIG. 26 is configured such that the log acquiring unit 11 acquires, as the log indicating a change in the body of the person to be diagnosed, a log indicating a change in a sleeping state of the person to be diagnosed, and the environment data acquiring unit 15 acquires environment data indicating an operation status of an air conditioner as the environment data indicating an environment around the person to be diagnosed, and includes a display data generating unit 73 to generate display data for displaying the change in the sleeping state indicated by the log acquired by the log acquiring unit 11 and the operation status of the air conditioner indicated by the environment data acquired by the environment data acquiring unit 15 on the screen. Therefore, similarly to the presymptomatic disease diagnosis device illustrated in FIG. 1, the presymptomatic disease diagnosis device illustrated in FIG. 26 can diagnose a presymptomatic disease in the abnormal finding absent state, and can check the relationship between a change in the sleeping state of the person to be diagnosed and an operation status of the air conditioner.

The presymptomatic disease diagnosis device illustrated in FIG. 11, 16, 20, 23, or 26 includes a log acquiring unit 11 and an environment data acquiring unit 15. The presymptomatic disease diagnosis device may include a data transmission unit (not illustrated), and the data transmission unit may transmit each of the log acquired by the log acquiring unit 11 and the environment data acquired by the environment data acquiring unit 15 to an external device. For example, if the external device is a device managed by a maintenance company, when the device receives each of the log and the environment data transmitted from the presymptomatic disease diagnosis device, an employee or the like of the maintenance company can check whether or not cleaning of a sensor that collects the log or a sensor that collects the environment data is necessary, whether or not replacement of the filter in the sensor is necessary, or the like.

The presymptomatic disease diagnosis device 1 according to the first to sixth embodiments includes the log acquiring unit 11 and the nursing care data acquiring unit 12. The presymptomatic disease diagnosis device may include a data transmission unit (not illustrated), and the data transmission unit may transmit each of the log acquired by the log acquiring unit 11 and the nursing care data acquired by the nursing care data acquiring unit 12 to an external device. If the external device is, for example, a device managed by a hospital or a device managed by a pharmacy, when the device receives each of the log and the nursing care data transmitted from the presymptomatic disease diagnosis device, the doctor or the like can determine the necessity of diagnosis for the person to be diagnosed, the necessity of prescription for the person to be diagnosed, the necessity of nursing care for the person to be diagnosed, or the like.

The presymptomatic disease diagnosis device 1 according to the first to sixth embodiments may transmit data acquired from the outside and diagnostic data acquired from the trained models 43 and 46 to an external device (not illustrated). The data acquired from the outside is a log, nursing care data, environment data, or vital data.

As a result, a company or the like that handles an external device (not illustrated) can utilize data transmitted from the presymptomatic disease diagnosis device 1 for business or the like.

In addition, the presymptomatic disease diagnosis device 1 may predict a risk of the person to be diagnosed from the data acquired from the outside and the diagnostic data acquired from the trained models 43 and 46, and transmit prediction data indicating the risk to an external device (not illustrated).

The presymptomatic disease diagnosis device 1 according to the first to sixth embodiments may monitor the behavior of the person to be diagnosed on the basis of the position information output from the GPS sensor in a case where the person to be diagnosed carries a GPS sensor, and may issue an alarm in a case where the behavior of the person to be diagnosed is different from the usual behavior of the person to be diagnosed.

As the behavior different from usual, for example, a case is assumed where the walking speed of the person to be diagnosed is slower than the usual walking speed, and the rate of the slower walking speed is larger than a preset reference value.

In addition, as the behavior different from usual, for example, a case is assumed where erroneous fastening of buttons in clothes worn by the person to be diagnosed is found for a plurality of days.

Note that, in the present disclosure, it is possible to freely combine each embodiment, to modify arbitrary components of each embodiment, or to omit arbitrary components in each embodiment.

### INDUSTRIAL APPLICABILITY

The present disclosure relates to a presymptomatic disease diagnosis device, a presymptomatic disease diagnosis method, and a trained model generation device.

### REFERENCE SIGNS LIST

1: presymptomatic disease diagnosis device, 2: display device, 3: trained model generation device, 11: log acquiring unit, 12: nursing care data acquiring unit, 13: presymptomatic disease diagnosing unit, 14: display processing unit, 15: environment data acquiring unit, 15a-1 to 15a-N: sensor, 16: presymptomatic disease diagnosing unit, 17: vital data acquiring unit, 18: determination unit, 19: display data generating unit, 21: log acquiring circuit, 22: nursing care data acquiring circuit, 23: presymptomatic disease diagnosing circuit, 24: display processing circuit, 25: environment data acquiring circuit, 26: presymptomatic disease diagnosing circuit, 27: vital data acquiring circuit, 28: determination circuit, 29: display data generating circuit, 31: memory, 32: processor, 41: data acquiring unit, 42: trained model generating unit, 43: trained model, 44: data acquiring unit, 45: trained model generating unit, 46: trained model, 51: data acquiring circuit, 52: trained model generating circuit, 53: data acquiring circuit, 54: trained model generating circuit, 61: memory, 62: processor, 71: skeleton analysis unit, 72: display data generating unit, 73: display data generating unit, 81: skeleton analysis circuit, 82: display data generating circuit, 83: display data generating circuit

1. A presymptomatic disease diagnosis device comprising:
processing circuitry performing a process to:
acquire a log indicating a change in a body of a person to be diagnosed;
acquire nursing care data indicating a nursing care content for the person to be diagnosed; and
give the log acquired and the nursing care data acquired to a trained model and acquire, from the trained model, diagnostic data indicating presymptomatic diseases including a state of no diagnosis of there being abnormality, possibly occurring in the person to be diagnosed.

2. The presymptomatic disease diagnosis device according to claim 1, wherein
the process acquires, as the log indicating a change in the body of the person to be diagnosed, a log indicating a change in a sleeping state of the person to be diagnosed,
the process acquires, as the nursing care data indicating a nursing care content for the person to be diagnosed, nursing care data in which whether or not the person to be diagnosed takes sleeping medication or an amount of exercise of the person to be diagnosed is recorded, and
the process gives the log acquired and the nursing care data acquired to the trained model, and acquires diagnostic data indicating a pre-stage state of insomnia as the presymptomatic disease from the trained model.

3. The presymptomatic disease diagnosis device according to claim 1, wherein
the process acquires, as the log indicating a change in the body of the person to be diagnosed, a log indicating a state change during a meal in the person to be diagnosed,
the process acquires, as the nursing care data indicating a nursing care content for the person to be diagnosed, nursing care data in which meal contents of the person to be diagnosed are recorded, and
the process gives the log acquired and the nursing care data acquired to the trained model, and acquires diagnostic data indicating a pre-stage state of aspiration pneumonia as the presymptomatic disease from the trained model.

4. The presymptomatic disease diagnosis device according to claim 1, wherein
the process acquires, as the log indicating a change in the body of the person to be diagnosed, a log indicating a change in a walking state of the person to be diagnosed,
the process acquires, as the nursing care data indicating a nursing care content for the person to be diagnosed, nursing care data in which a walking amount of the person to be diagnosed is recorded, and
the process gives the log acquired and the nursing care data acquired to the trained model, and acquires diagnostic data indicating a pre-stage state of a walking disorder as the presymptomatic disease from the trained model.

5. The presymptomatic disease diagnosis device according to claim 1, wherein
the process acquires, instead of the log indicating a change in the body of the person to be diagnosed, a log indicating an operation history of a device by the person to be diagnosed,
the process acquires, as the nursing care data indicating a nursing care content for the person to be diagnosed, nursing care data in which an erroneous operation of a device by the person to be diagnosed is recorded, and
the process gives the log acquired and the nursing care data acquired to the trained model, and acquires diagnostic data indicating that it is a pre-stage state of dementia as the presymptomatic disease from the trained model.

6. The presymptomatic disease diagnosis device according to claim 1, the process further comprising to acquire environment data indicating an environment around the person to be diagnosed, wherein
the process gives the log acquired, the nursing care data acquired, and the environment data acquired to the trained model, and acquires diagnostic data indicating the presymptomatic disease from the trained model.

7. The presymptomatic disease diagnosis device according to claim 6, the process further comprising to compare boundary data indicating a boundary between a normal environment and an abnormal environment around the person to be diagnosed with the environment data acquired, and determines whether the environment around the person to be diagnosed is a normal environment or an abnormal environment on a basis of a comparison result between the boundary data and the environment data.

8. The presymptomatic disease diagnosis device according to claim 1, the process further comprising:
to acquire vital data indicating vitals of the person to be diagnosed; and
to compare the vital data acquired with a threshold value, and determine whether the vitals of the person to be diagnosed are normal or abnormal on a basis of a comparison result between the vital data and the threshold value.

9. The presymptomatic disease diagnosis device according to claim 1, the process further comprising:
to acquire vital data indicating vitals of a staff who cares for the person to be diagnosed; and
to compare the vital data acquired with a threshold value, and determine whether the vitals of the staff are normal or abnormal on a basis of a comparison result between the vital data and the threshold value.

10. The presymptomatic disease diagnosis device according to claim 6, wherein
the process acquires environment data indicating an observation result of an environment from each of a plurality of sensors that observes an environment around the person to be diagnosed, and
the process of the presymptomatic disease diagnosis device further comprises to generate display data for displaying a position where each of the sensors is installed and environment data output from each of the sensors on a screen.

11. The presymptomatic disease diagnosis device according to claim 1, wherein
the process acquires, as the log indicating a change in the body of the person to be diagnosed, video data of a camera photographing the person to be diagnosed, and
the process further comprises:
to analyze movement of a skeleton of the person to be diagnosed from the video data acquired, and output skeleton data indicating the movement of the skeleton of the person to be diagnosed; and
to generate display data for displaying the movement of the skeleton of the person to be diagnosed on a screen according to the skeleton data output.

12. The presymptomatic disease diagnosis device according to claim 6, wherein
the process acquires, as the log indicating a change in the body of the person to be diagnosed, a log indicating a change in a sleeping state of the person to be diagnosed,
the process acquires environment data indicating an operation status of an air conditioner as the environment data indicating an environment around the person to be diagnosed, and
the process of the presymptomatic disease diagnosis device further comprises to generate display data for displaying the change in the sleeping state indicated by the log acquired and the operation status of the air conditioner indicated by the environment data acquired on a screen.

13. A presymptomatic disease diagnosis method comprising:
acquiring a log indicating a change in a body of a person to be diagnosed;
acquiring nursing care data indicating a nursing care content for the person to be diagnosed; and
giving the log acquired and the nursing care data acquired to a trained model and acquiring, from the trained model, diagnostic data indicating presymptomatic diseases including a state of no diagnosis of there being abnormality, possibly occurring in the person to be diagnosed.

14. A trained model generation device comprising:
processing circuitry performing a process to:
acquire a log indicating a change in a body of a person to be diagnosed, acquire nursing care data indicating a nursing care content for the person to be diagnosed, and acquire teacher data indicating a presymptomatic diseases including a state of no diagnosis of there being abnormality, possibly occurring in the person to be diagnosed or teacher data indicating not a presymptomatic disease; and
to learn the presymptomatic diseases by using each of the log, the nursing care data, and the teacher data acquired, and generate a trained model that outputs diagnostic data indicating the presymptomatic diseases including a state of no diagnosis of there being abnormality, possibly occurring in the person to be diagnosed when the log indicating a change in the body of the person to be diagnosed and the nursing care data indicating a nursing care content for the person to be diagnosed are given.

15. The trained model generation device according to claim 14, wherein
the process acquires a log indicating a change in a body of a person to be diagnosed, acquires nursing care data indicating a nursing care content for the person to be diagnosed, acquires environment data indicating an environment around the person to be diagnosed, and acquires teacher data indicating a presymptomatic diseases including a state of no diagnosis of there being abnormality, possibly occurring in the person to be diagnosed or teacher data indicating not a presymptomatic disease, and
the process learns the presymptomatic diseases by using each of the log, the nursing care data, and the teacher data acquired, and generates a trained model that outputs diagnostic data indicating the presymptomatic diseases including a state of no diagnosis of there being abnormality, possibly occurring in the person to be diagnosed when the log indicating a change in the body of the person to be diagnosed, the nursing care data indicating a nursing care content for the person to be diagnosed, and the environment data indicating an environment around the person to be diagnosed are given.
